(19) United States
(12) Patent Application Publication
(10) Pub. No.: US 2019/0328312 A1
(43) Pub. Date: Oct. 31, 2019

(54) APPARATUS AND METHOD FOR IMAGING AND MODELING THE SURFACE OF A THREE-DIMENSIONAL (3-D) OBJECT

(71) Applicant: James E SCHROEDER, San Antonio, TX (US)

(72) Inventor: James E SCHROEDER, San Antonio, TX (US)

(73) Assignee: The Board of Regents of the University of Texas System, Austin, TX (US)

(21) Appl. No.: 16/310,704

(22) PCT Filed: Jun. 19, 2017
(86) PCT No.: PCT/US2017/038137

§ 371 Date: Dec. 17, 2018

Related U.S. Application Data
(60) Provisional application No. 62/351,699, filed on Jun. 17, 2016.

(51) Int. Cl. A61B 5/00; A61F 2/78; G02B 13/06; G02B 17/06; G06T 11/00

(52) U.S. Cl. CPC A61B 5/444; A61F 2/78; G02B 13/06; G06T 11/00; A61B 5/443; A61B 5/0075; G02B 17/06

(57) ABSTRACT

Certain embodiments are directed to methods, devices, and/or systems for viewing and imaging all or most of the surface area of a three-dimensional (3-D) object with one or more two-dimensional (2-D) images.

### CROSS-REFERENCE TO RELATED APPLICATIONS

This application claims priority to U.S. Provisional Application No. 62/351,699, filed Jun. 17, 2016, which is incorporated by reference in its entirety.

### FIELD OF THE INVENTION

Embodiments described herein are related to the field of imaging and to the uses thereof, especially in quality control, capturing a large portion or all of the entire surface area of a three-dimensional (3-D) object on one or more two-dimensional (2-D) images, using the location of a point-of-interest found on the one or more 2-D images to specify the location of that point on the surface of the 3-D object, and using the one or more 2-D images to create a virtual or real 3-D model of the 3-D object. Of particular interest is when the image(s) display the visible or infrared portions of the electromagnetic energy continuum, and their use in medicine and health care, especially in prosthetics and orthotics.

### BACKGROUND

The number of amputations performed has risen over the past two decades partly due to complications associated with vascular disorders in the nation's increasing diabetic population (dysvascular population) (Centers for Disease Control and Prevention Web Site (May 17, 2016). Number (in Millions) of Civilian, Non-Institutionalized Persons with Diagnosed Diabetes, United States, 1980-2014, available on the world wide web at cdc.gov/diabetes/statistics/prev/national/figpersons.htm) and partly due to casualties from recent military conflicts or other traumatic events (traumatic population) (DePalma et al., (2005) New England Journal of Medicine, 352: 1335-42.). The majority of amputations are unilateral and occur below the knee (transtibial) [World Health Organization. (2004). The rehabilitation of people with amputations. United States Department of Defense, Moss Rehabilitation Program, Moss Rehabilitation Hospital, USA, available online May 17, 2016 at docplayer. net/960920-The-rehabilitation-of-people-with-amputations html; Smith and Ferguson, (1999), Clinical Orthopedic Relational Research, 361:108-15.]. Most amputees wear a prosthetic device, usually comprising a custom-fit socket, a form of suspension to hold the socket in place on the residual limb, and a prosthetic foot. For patients with a prosthetic lower limb, obtaining/maintaining an excellent fit and proper adjustment for their prosthesis is critical for the long-term health of both the residual and sound limbs. This is especially true for the dysvascular population which is known to be susceptible to skin-related health problems (Lyon et al., (2000), Journal of the American Academy of Dermatology, 42:501-7) on both their sound and residual limbs, and for whom the interface between the prosthetic socket and the residual limb is a site of potentially harmful pressure [Houston et al., (2000), RESNA Proceedings—2000, Orlando, Fla.; Herrman et al., (1999), Journal of Rehabilitation Research & Development, 36(2):109-20; Colin and Saumet, (1996), Clinical Physiology, 16(1):61-726-8] that can produce skin irritation that can develop into a lesion. The common presence of sensory neuropathy in this population further reduces the chances of early detection, and makes the work of the prosthetist even more difficult (e.g., patients often are not able to sense/report problem areas). Also, once a problem develops, healing can be a slow process because of the patient's vascular problems. In addition to creating health issues, a poorly fitted prosthesis often leads to its abandonment by the user, potentially impacting that person's overall mobility and quality of life.

There remains a need for additional devices and methods for measurement and assessment tools to achieve the best possible fit for a prosthetic or orthotic device.

### SUMMARY

Disclosed herein is an imaging technology that allows imaging of a large portion or the entire surface of a 3-D object. This imaging technology may be standardized with respect to capturing 3-D spatial information related to the surface of a physical object in one or more 2-D images. Examples of spatial information include shades of gray (e.g., when a black-and-white still-frame photographic, movie, or video camera are used); different colors (e.g., when a color still-frame photographic, movie, or video camera are used); temperature (e.g., when a thermographic camera is used); ultraviolet wavelength (e.g., when ultraviolet camera is used); color and distance (e.g., when a light-field camera is used); and other ranges of the electromagnetic spectrum (as corresponding cameras or devices are available). To further contribute to its usefulness, information from multiple energy dimensions can be obtained for the same viewed object and mixed/overlaid to facilitate interpretation. For example, in healthcare applications, the photographic and thermographic images of an affected portion of the body can be combined or overlaid to help the healthcare provider interpret the image.

In one representative embodiment, the technology described can be applied to imaging an amputee's amputated (residual) and/or sound limbs and helping a healthcare provider identify and document locations of concern at which there is visible or thermal evidence of sores or early signs of skin irritation that could be indicative of rubbing or pressure points. Regions of increased heat (relatively high peripheral blood flow) and/or regions of decreased heat (relatively poor peripheral blood flow) could implicate health concerns. In some instances, the imaging technology may use infrared imaging (thermography) to identify and document locations of concern. In some instances, locations of concern on a subject (i.e., a person, animal, object, or any portion thereof that is of interest) may be used to assess the fit of a prosthetic limb or orthotic device. This information, when provided to a prosthetist or orthotist, can be used to determine the corresponding regions in a prosthesis or orthosis that need modification to avoid more significant future irritation due to the prosthesis or orthosis (e.g., skin ulcers). In some instances, the imaging technology may use more conventional photographic or video imaging to identify and document existing locations of concern. This information also can be used by a prosthetist or orthotist to modify a prosthesis or orthosis to avoid irritation due to the prosthesis or orthosis. In certain aspects, assessment can be performed during a single appointment or session while the patient is being fitted for a prosthesis or orthosis. In some aspects, this imaging apparatus and method may be used in monitoring the limb health of a residual limb or the contralateral unaffected limb at all stages of a disease or condition. The imaging apparatus and method may be used to detect areas of concern before amputation that with medical intervention could reduce the need for amputation. The tools and methods disclosed may be standardized with respect to the size, degree of irritation, and location of problem areas.

Another representative embodiment relates to the use of thermography in quality control and maintenance. Faulty solder joints and electronic devices such as power utility transformers about to fail often have distinctive heat signatures which can be observed and documented at a distance using thermal cameras. Such procedures could be substantially improved if the camera were able to capture most or all of the surface of the 3-D mechanism/component being assessed. Not only would such an enhancement increase the likelihood of detecting a problem, but it also could be used to identify the precise location of the problem, perhaps indicating which specific component or sub-circuit is involved.

In addition to capturing and storing surface-related information from an imaged 3-D object, information about the size and shape of the object being imaged (which can be obtained using a variety of methods—see below) can be combined to the surface information using special image processing software to create virtual or real (e.g., using 3-D printing, selective laser sintering device, etc.) models of the object. Hence, another representative embodiment relates to the use of combining captured information about the image of a surface of a 3-D object with size and shape information about that object to create virtual or real models of the imaged object.

In some aspects, a three-dimensional imaging system for producing a two-dimensional image of a physical object is disclosed herein. In some aspects, the system includes a reflective surface that reflects at least one portion of the electromagnetic spectrum and at least one camera facing the reflective surface that is capable of capturing at least one image based on reflected electromagnetic radiation, wherein (i) the reflective surface facing at least one camera is concave, comprises an apex, and is configured to reflect at least one type of electromagnetic radiation emanating or reflecting from the surface of a physical object positioned along the principal axis of the reflective surface and (ii) at least one camera or imaging device positioned to capture the emitted or reflected electromagnetic radiation. In some aspects the imaging system disclosed herein further contains a computer based image processor wherein the computer based image processor is configured to determine the location on or the portion of the physical object that is emitting or reflecting the electromagnetic radiation that is being received by the at least one camera. In some aspects the concave surface is spherical, conical, or parabolic. In some aspects the concave surface contains more than one shape. In some aspects the concave surface contains a conical surface portion with a first reflective angle more distant from the apex of the reflective surface and a conical and/or spherical surface portion having a second portion with an increased reflective angle that is closer or proximal to the base of the reflective surface. In some aspects the concave surface is configured to reflect radiation emanating or reflecting from a physical object along the principal axis and 360 degrees about the principle axis. In some aspects the reflective surface is capable of reflecting more than one type of electromagnetic radiation. In some aspects at least one camera contains a fisheye lens. In some aspects at least one camera is capable of capturing the surface image of the object as a single image. In some aspects a computer based image processor is configured to provide a representative view of the object surface and the representative view can be manipulated in three dimensions. In some aspects the system is capable of capturing the surface image of the object from two or more angles from the principle axis of the reflective surface, from two or more distances from the apex of the reflective surface, and/or using two or more focal distances. In some aspects the computer based image processor is configured to determine and/or assign a size and/or shape to a location on the physical object that is emitting the reflected electromagnetic radiation. In some aspects at least one camera is capable of capturing multiple types of electromagnetic radiation and/or the imaging system comprises at least two cameras each that are capable of capturing a different type of electromagnetic radiation than the other. In some aspects at least one type of electromagnetic radiation is infrared light and at least one camera is a thermographic camera responsive to the infrared energy spectrum. In some aspects the concave surface is aluminum. In some aspects the concave surface is highly polished aluminum. In some aspects the system is configured to produce an image that is a hotspot map of the object. In some aspects the system is configured to produce an image that is a cold-spot map of the object.

Certain aspects are directed to a computer based image processor capable of mapping a location on an object based on a reflection of the object from a concave reflector, the reflection being captured by at least one camera. In some aspects the location mapped is a hotspot on an object. In some aspects the location mapped is a cold-spot on an object. In some aspects the processor is further capable of providing a representative view of the object surface, wherein the representative view can be manipulated in three dimensions. In some aspects the processor is further capable of determining and/or assigning a size and/or shape to a location or position on the object. In some aspects the processor is capable of overlaying (i) representations of multiple types of electromagnetic energy on the map of the object or (ii) representations of one or more types of electromagnetic energy and a size and/or shape on the map of the object.

Further aspects are directed to a computer based image processor capable of creating a panoramic map of an object based on a reflection of an object from a concave reflector captured by at least one camera. In some aspects the computer based image processor is capable of mapping a location on the panoramic map. In some aspects the location mapped is a hotspot on an object. In some aspects the location mapped is a cold-spot on an object. In some aspects the processor is further capable of providing a panoramic map that can be manipulated in three dimensions. In some aspects the processor is further capable of determining and/or assigning a size and/or shape to a location/position on the object. In some aspects the processor is capable of overlaying (i) representations of multiple types of electromagnetic energy on the map of the object or (ii) representations of one or more types of electromagnetic energy and a size and/or shape on the map of the object.

Certain aspects are directed to methods for representing a three-dimensional object by any of the computer based image processors disclosed herein. In some aspects the computer based image processor produces a two-dimensional map of at least one image taken by at least one camera of a reflection of the object off of a concave surface. In some aspects, the three-dimensional object is an organism or part of an organism. In some aspects the three-dimensional object is a residual portion of an amputation. In some aspects, the three-dimensional object is an electronic device, a portion of an electronic device, or a component of an electronic device. In some aspects the reflective concave surface reflects infrared radiation. In some aspects the reflective concave surface reflects visible light. In some aspects the reflective concave surface reflects multiple types of electromagnetic energy. In some aspects the reflective concave surface reflects infrared radiation and visible light. In some aspects, the method further includes determining and/or assigning a size and/or shape to a location on the three-dimensional object. In some aspects the method further includes overlaying (i) representations of multiple types of electromagnetic energy on the representation of the three-dimensional object or (ii) representations of one or more types of electromagnetic energy and a size and/or shape on the representation of the three-dimensional object.

Other aspects are directed to methods for representing a three-dimensional object by any of the computer based image processors disclosed herein. In some aspects, the computer based image processor produces a three-dimensional map of at least one image taken by at least one camera of a reflection of the object off of a concave surface. In some aspects, the three-dimensional object is an organism or part of an organism. In some aspects the three-dimensional object is a residual portion of an amputation. In some aspects, the three-dimensional object is an electronic device, a portion of an electronic device, or a component of an electronic device. In some aspects the reflective concave surface reflects infrared radiation. In some aspects the reflective concave surface reflects visible light. In some aspects the reflective concave surface reflects multiple types of electromagnetic energy. In some aspects the reflective concave surface reflects infrared radiation and visible light. In some aspects, the method further includes determining and/or assigning a size and/or shape to a location on the three-dimensional object. In some aspects the method further includes overlaying (i) representations of multiple types of electromagnetic energy on the representation of the three-dimensional object or (ii) representations of one or more types of electromagnetic energy and a size and/or shape on the representation of the three-dimensional object.

Certain aspects are directed to methods for representing a three-dimensional structure of a physical object as a representation that can be manipulated in three-dimensions. In some aspects, the method includes placing at least a portion of the physical object along the principal axis in front of a reflective concave surface and positioning at least one camera to capture the reflection from the reflective concave surface and capturing and processing at least one image of the physical object based on the reflection from the concave surface. In some aspects, the method further includes determining and/or assigning a size and/or shape to a location on the physical object. In some aspects the method further includes mapping the captured reflection to a physical object being imaged using a computer based image processor. In some aspects the method further includes overlaying (i) representations of multiple types of electromagnetic energy on the representation of the physical object or (ii) representations of one or more types of electromagnetic energy and a size and/or shape on the representation of the physical object. In some aspects the representation is created using only one or two captured images comprising the reflection from the concave surface. In some aspects, the physical object is an organism or part of an organism. In some aspects the physical object is a residual portion of an amputation. In some aspects, the physical object is an electronic device, a portion of an electronic device, or a component of an electronic device. In some aspects the reflective concave surface reflects infrared radiation. In some aspects the reflective concave surface reflects visible light. In some aspects the reflective concave surface reflects multiple types of electromagnetic energy. In some aspects the reflective concave surface reflects infrared radiation and visible light.

Certain embodiments are directed to methods for representing a three-dimensional structure of a physical object in a two-dimensional map. In some aspects, the method includes placing at least a portion of the physical object along the principal axis in front of a reflective concave surface and positioning at least one camera to capture the reflection from the reflective concave surface, and capturing and processing at least one image of the physical object based on the reflection from the concave surface. In some aspects the method further includes determining and/or assigning a size and/or shape to a location on the physical object. In some aspects the method further includes mapping the captured reflection to a location on the part of the physical object being imaged using a computer based image processor. In some aspects the method further includes overlaying (i) representations of multiple types of electromagnetic energy on the representation of the physical object or (ii) representations of one or more types of electromagnetic energy and a size and/or shape on the representation of the physical object. In some aspects the representation is created using only one or two captured images comprising the reflection from the concave surface. In some aspects, the physical object is an organism or part of an organism. In some aspects the physical object is a residual portion of an amputation. In some aspects, the physical object is an electronic device, a portion of an electronic device, or a component of an electronic device. In some aspects the reflective concave surface reflects infrared radiation. In some aspects the reflective concave surface reflects visible light. In some aspects the reflective concave surface reflects multiple types of electromagnetic energy. In some aspects the reflective concave surface reflects infrared radiation and visible light.

Certain aspects are directed to methods of identifying the location of skin irritation and/or early signs of skin irritation on a subject. In some aspects the method includes placing a portion of the subject to be imaged, the subject having actively worn a prosthetic or orthotic device, along the principal axis of a reflective concave structure in view of at least one camera connected to an imaging system, capturing at least one image of reflected infrared radiation emitted from the part of the subject being imaged with the at least one camera, mapping the captured infrared reflection to a location on the part of the subject being imaged using a computer based image processor, and identifying skin irritation as the location emitting infrared irradiation or an increased level of infrared irradiation as compared to a reference. In some aspects the method further includes, capturing at least one image of reflected visible light emitted from the part of the subject being imaged with the at least one camera, mapping the reflected visible light to a location on the part of the subject being imaged using a computer based image processor, and overlaying the infrared reflection and the visible light mapping in a representation of the part of the subject being imaged. In some aspects the method further includes determining and/or assigning a size and/or shape to the location on the part of the subject being imaged. In some aspects, the part of the subject imaged includes a residual portion of an amputation. In some aspects the method further includes imaging the subject before the subject wears a prosthetic or orthotic device (e.g., obtaining a reference) and imaging the subject after the subject has worn the device. In some aspects, the method further includes modifying or adjusting the prosthetic or orthotic device to create a better goodness-of-fit for the device based on the location of increased and or decreased infrared radiation.

Other embodiments of the invention are discussed throughout this application. Any embodiment discussed with respect to one aspect of the invention applies to other aspects of the invention as well and vice versa. Each embodiment described herein is understood to be an embodiment of the invention that is applicable to all aspects of the invention. It is contemplated that any embodiment discussed herein can be implemented with respect to any method or composition of the invention, and vice versa. Furthermore, compositions and kits of the invention can be used to achieve methods of the invention.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1. (left) Standard view of a Rubik's Cube; (right) same Rubik's cube viewed inside a concave surface that reflects visible light.

FIG. 2. (left) Non-limiting representation of a cylindrical test object which has a star on the nearest end and concentric circles on its outer surface which are equally spaced along the cylinder's longitudinal axis; (right) that same cylindrical test object as imaged when positioned inside a representative conical viewing chamber or reflective surface with the cone's apex angle (angle of the cone's reflective surface relative to the focal axis, in this case, also the cylinder's longitudinal axis), the frontal focal length (distance from the viewing eye/camera lens to the cone's apex), and the camera's viewing angle jointly adjusted to produce a nearly longitudinally-perfect perpendicular view of the outer walls of the test object (i.e., the equally spaced lines on the outer wall of the object are depicted as equally spaced concentric circles when viewed on the reflective surface).

FIG. 3 (left) Non-limiting representation of a cylindrical test object which has circular decals of equal radius positioned on its exterior surface (at four latitudinal locations and three longitudinal locations); (right) that same cylindrical test object as imaged when positioned inside a representative conical viewing chamber or reflective surface with the cone's apex angle (angle of the cone's reflective surface relative to the focal axis, in this case, also the cylinder's longitudinal axis), the frontal focal length (distance from the viewing eye/camera lens to the cone's apex), and the camera's viewing angle jointly adjusted to produce a nearly longitudinally-perfect perpendicular view of the outer walls of the test object. Note that unlike the longitudinal aspects, the latitudinal aspects of the reflected image are systematically distorted, with the circular “face” decals nearer to the camera “magnified” relative to those farther away. As described in the text, the lateral distortions can be removed using special image processing software which systematically analyzes rays comprising individual pixels (as shown in A) or rays comprising angular partitions (as shown in B).

FIG. 4 Non-limiting representation of an apparatus for recording 3-D information of an amputee's residual limb with one 2-D image. As discussed in the text, the residual limb may be imaged using an infrared camera for medical reasons.

FIG. 5 Non-limiting representation showing a utility company worker using a thermal camera (A) to remotely assess the operation of a transformer (B) on a utility pole which has been positioned inside a concave thermally reflective surface (C) in an orientation that allows the worker to assess a large portion of the transformer's external surface in search of hot or cold regions which are known to be early indicators of device failure.

FIG. 6 3-D image of the first subject's residual limb after a 20-min initial rest period and before walking. The green area inside the smaller blue circle is the end of the subject's residual limb. Area A is a hotspot directly visible (because the subject's leg is tilted downward toward the camera); region B is the same area reflected of the sides of the conical viewing chamber.

FIG. 7 Initial thermal (left) and LD (right) images of the anterior view before walking.

FIG. 8 Standard thermal (left) and LD (right) images of the selected ROI for Subject 1 (the same general area of increased heat shown in Region A of FIG. 6 and the anterior thermal image shown in FIG. 7.

FIG. 9 Initial 3-D image before walking during the second session. The ROI identified in the first session is still evident (the subject's limb is better oriented (more in line with the camera) than was the case in the first session (FIG. 20), and all three fiducials positioned on the tibial crest are visible and generally aligned.

FIG. 10 Thermal (left) and LD (right) images of the anterior view of Subject 1's residual limb after the initial rest period in the second session. The identified ROI is salient in the thermograph but not evident in the LD image.

FIG. 11 Thermal images of the ROI identified in the first session before any walking (left) and after the 100 m walk (right).

FIG. 12 LD images of the ROI identified in the first session before any walking (left) and after the 100 m walk (right).

FIG. 13 Map of mean peak plantar pressures while walking 100 m (left), corresponding thermal image (center) and LD image (right) of the bottom of Subject 1's sound foot after walking 100 m in the second session.

FIG. 14 3-D image of the second subject's residual limb following the 50 m walk. The arrow points to the area that was designated as the primary ROI for Subject 2—note like the primary ROI for Subject 1 it is located near the tibial crest, but unlike Subject 1, it is much more proximal—located above the most proximal marker and nearer to the knee.

FIG. 15 Initial, pre-walk anterior standard thermal (left) and LD (right) images of the second subject's residual limb. Note that, unlike the first subject, the thermal and LD images both show increased measures in the area associated with the ROI.

FIG. 16 Thermal images of the ROI for Subject 2 after 50(left) and 100 m(right) walks.

FIG. 17 LD images of the ROI for Subject 2 after 50(left) and 100 m(right) walks. Unlike the ROI identified for Subject 1, the LD images are measuring patterns of perfusion that are highly similar for the two walks and consistent with the thermal measures (FIG. 16).

FIG. 18 3-D image taken at the beginning of the second session; note the ROI absence.

FIG. 19 Anterior thermal (left) and LD (right) anterior images; note the absence of the ROI.

FIG. 20 Standard thermal images of the ROI identified in the first session before walking (left), after walking 50 m (center), and after walking 100 m (right) in the second session; note the absence of the concentrated site evident in the first session.

FIG. 21 Standard LD images of the ROI identified in the first session before walking (left), after walking 50 m (center), and after walking 100 m (right) in the second session; note the absence of the concentrated site evident in the first session.

FIG. 22 Map of mean peak plantar pressures while walking 100 m (left), corresponding thermal image (center) and LD image (right) of the bottom of Subject 2's sound foot after walking 100 m in the first session.

FIG. 23 Depiction of rays in the viewing chamber for an observer/camera located at point b and cone vertex located at point 1. The paths of rays for 4 points observed on the major axis between a and b are depicted; the paths differ by equally separated viewing angles (bce, bfg, bhi, and bjk).

### DESCRIPTION

Embodiments of the current invention can be applied in a variety of settings to image virtually any object by using any camera or device capable of capturing and displaying an array of measures sensitive to a selected range of the electromagnetic continuum. The visible light spectrum and the infrared range of electromagnetic energy were selected as example applications; the light spectrum was selected because it provides the most illustratable examples and the infrared continuum was selected because of its common use in quality control settings (e.g., to identify faulty solder joints or electronic components about to fail) and because of its use in medical settings (e.g., to detect areas of the skin with relatively high or low peripheral blood circulation). Regarding medical applications, the general field of prosthetics and orthotics was selected as the primary example setting because it provides a reasonable, representative, and understandable embodiment which illustrates the invention's use and usefulness.

Goodness-of-fit (GoF) for a prosthesis has been shown to be a prominent concern for amputees and the medical community that serves them. Legro et al. (1998, Archives of Physical Medicine & Rehabilitation, 79(8):931-38) identified several factors contributing to patient satisfaction, which included the goodness of socket fit. Sherman (1999, Journal of Rehabilitation Research & Development, 36(2):100-08) noted that 100% of his US veteran sample reported having problems using their prosthesis for work, with most problems associated with the attachment methods. Sherman also reported that 54% of his patient sample did not use their prosthesis because of pain, discomfort, or poor fit. Bhaskaranand et al. (2003, Archives of Orthopaedic & Trauma Surgery, 123(7):363-66) reported that reasons cited for not using upper-extremity prostheses included poor fit. Klute et al. (2009, Journal of Rehabilitation Research & Development, 46(3):293-304) conducted a focus group at the VA's Center of Excellence in Prosthetics to assess the needs of veteran amputees wearing prosthetic devices and reported: “While generally positive about their mobility, all prosthetic users had difficulties or problems at all stages in the processes of selecting, fitting . . . ” Gailey et al. (2008, Journal of Rehabilitation Research & Development, 45(1):15-30) reported that amputees “commonly complain of back pain, which is linked to poor prosthetic fit and alignment . . . .”

As disclosed herein, better imaging techniques can be used to help resolve many of these issues. Methods described herein use infrared imaging (thermography) to provide a cost-effective, non-invasive, safe, and affordable diagnostic/measurement tool. While the possibility of using thermography for detecting early signs of skin irritation from prostheses use has been noted, it is not being utilized. Transcutaneous oxygen pressure (TCPO₂) currently is widely used to obtain a reasonable measure of peripheral blood circulation, but instruments that measure TCPO₂ are restricted to measuring a single point on the surface of the limb. Laser-Doppler imaging (LDI) also provides a measure of peripheral circulation, and there are systems that can capture a 2-D image of an area of the skin, but a minimum of 5 images would have to be scanned (e.g., medial, lateral, anterior, posterior, and the end of the stump) to approach the level of information collected in one thermal image using this invention, and even then, the quality of information from the LDI would be suspect on the edges of the limbs because LDI is very sensitive to the distance from the object to the LDI sensor, and limbs have curved surfaces. In addition, the amount of time necessary to use LDI to scan five views of a limb is several times that required by a thermal camera in combination with the present invention.

Three-dimensional imaging can be expensive, requires a complex system, and requires a large amount of data to reproduce the 3-D image, which makes it difficult to transfer and store. Panoramic imaging (imaging of a 360° view of the surrounding environment) also can be expensive and require multiple images and/or a complex arrangement of lenses. In one approach to panoramic imaging, multiple images are captured of the panoramic view by multiple synchronized cameras or a single camera that is reoriented between shots. The combined imagines can then be combined and may need to be modified to fix distortions inherent in the system. In another approach to panoramic imaging, the entire panoramic view is captured in one frame. That system uses a complex and expensive lens system to capture a highly distorted image of a 360° panoramic view. To reproduce the panoramic view, the distortions are later removed by a complementary lens on a display system which projects the entire undistorted scene onto the walls of a circular theater or surface. That type of system was used by the United States military in the “Surface Orientation and Navigation Trainer” (SURNOT) to capture views of geographic sites (U.S. Pat. No. 4,421,486).

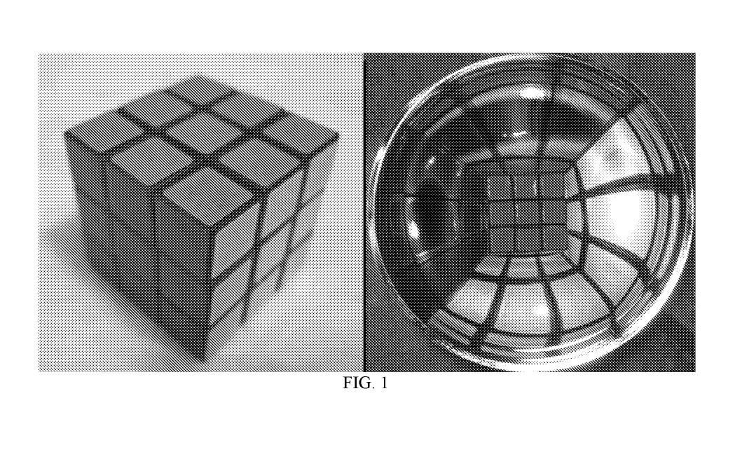

Certain embodiments of the present invention are directed to devices and methods that provide imaging technology that allows one or more 2-D images to capture a large portion or nearly the entire surface of a 3-D object. The images may include photographic images of everyday objects, thermographic images of an electronic device or component (such as when used in a quality control or maintenance setting), or thermographic images of an amputee's amputated (residual) limb and/or sound limb to assess the health of that limb or the GoF of a prosthetic device. With fiducial markers positioned at known locations on the object, or with additional information about the shape and size of the object, the location of a specific site (point or region) of interest identified in the 2-D image can be used to locate the corresponding site on the peripheral surface of the 3-D object by using common trigonometric or geometric functions and interpolation. Also, with additional information about the object's basic shape and dimensions (especially for simple geometric solid figures like cylinders, cones, cubes, pyramids, etc.), or with additional information about the size and shape of the object based on estimated distances from the camera to different sites on the object (e.g., using a light-field camera or some other distance-estimation technology—see the section below entitled “Size and Shape Information”), then special image analysis software can be used to create representative virtual models, or, when used in conjunction with a 3-D printer, selective laser sintering device, etc., create actual scaled models of that object. In the ‘Rubik's cube” example shown in FIG. 1, note that visual information about the “opposite side’ of the cube is not available; however, such information can be obtained by other means compatible with the current method; for example, by taking a second image after reversing the orientation of the object, or by increasing the radius of the viewing chamber or reflective surface, changing the angle of reflection for the reflective surface, and/or physically moving the cube toward the camera and away from the apex of the reflective surface (by suspension or by placing it on a pedestal). If the resulting 2-D image(s) is/are captured and stored in manipulable digital format, then special image processing software can be used to “wrap” the reflected surface information captured in the 2-D image(s), to a virtual 3-D representation of the object, such that size, shape, and appearance (visual, thermal, etc.,) are combined in the same virtual representation or when used in conjunction with a 3-D printer, selective laser sintering device, etc., create a corresponding scaled physical copy of the object.

Given the selection of a conical viewing chamber, the next task was to determine the best conical angle (angle between the cone's central axis (passing through the cone's vertex and the center of the camera's image), and the wall of the cone, with “best” defined as the angle that produced the most accurate perpendicular view. The thermal camera being used had a field-of-view of 28°, so the theoretical range of viewing angles for one side of the limb was from 0° to 14°; the real range is from about 3° to 14°, because the center of the image is the actual (non-reflected) distal end of the residual limb. Hence, the trigonometric question was—given the camera views from 3° to 14°, and the length of the object being viewed is about 42 cm long, then what reflective angle yields the most accurate perpendicular view? Another related question is, how long should the walls of the cone be to assure the entire limb is visible?

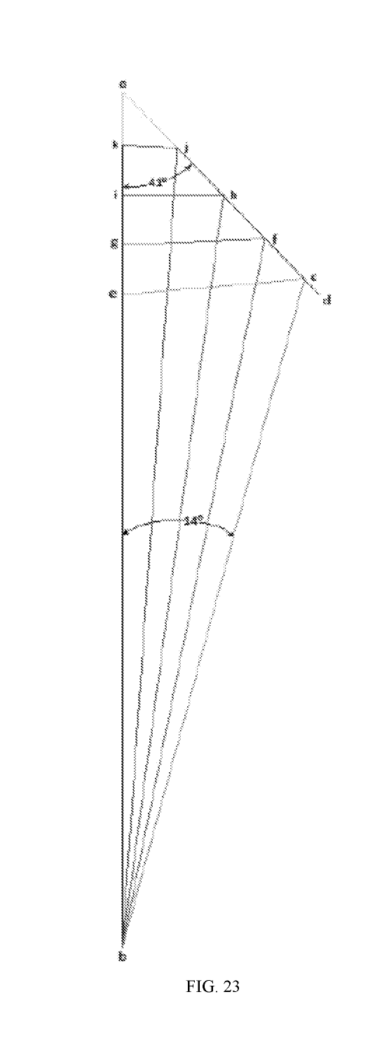

To address such questions, a spreadsheet was created which allows the following parameters to be manipulated to determine their overall effect on the reflected image: (1) distance from the observer/camera to the vertex of the viewing cone (FIG. 23); (2) the angle of the walls of the reflective cone (angle dab in FIG. 23); and (3) the “thickness” of the object being viewed (not shown in FIG. 23, but the perpendicular distance from line ab to the outer edge of the viewed object—for an amputee, the center of the tibia to the skin). After these variables are manually selected, the program produces a set of predicted points for the reflected object (each point indicates the distance from the vertex of angle dab in FIG. 23 to the point viewed on the major axis line ab) ranging from an observed angle of 3° to an observed angle of 14° in increments of 0.1°, along with corresponding descriptive statistics.

The equation is based on the fact that the reflective angle is specified, the distance between the observer and the cone's apex is specified, the viewing angle is specified (i.e., systematically varied from 3° to an observed angle of 14°), and is based on trigonometric functions—including the critical fact that the angle of incidence is equal to the angle of reflection.

A given estimated distance of a reflected point from the cone's vertex is computed by the following equation:

$$Distance from vertex ( a ) to viewed point on axis ab = ( ( ( X * Z ) / ( Y + Z ) ) ) - ( ( TAN ( RADIANS ( ( DEGREES ( ATAN ( ( ( X * Z ) / ( Y + Z ) ) / ( Y * ( ( X * Z ) / ( Y + Z ) ) ) ) ) ) - ( 180 - ( DEGREES ( ATAN ( ( ( X * Z ) / ( Y + Z ) ) / ( Y * ( ( X * Z ) / ( Y + Z ) ) ) ) ) ) - ( DEGREES ( ATAN ( ( X - ( ( X * Z ) / ( Y + Z ) ) ) / ( Y * ( ( X * Z ) / ( Y + Z ) ) ) ) ) ) ) ) ) ) * ( Y * ( ( X * Z ) / ( Y + Z ) ) ) ) \qquad Equation 1$$

where: X=distance ab in FIG. 23, Y=Tangent (Radians) of mirror angle (angle “dab” in FIG. 23), Z=Tangent (Radians) of observed angle (angle “abd” in FIG. 23).

Using this program, it was determined that a camera distance (ab) of 200 cm was adequate to capture the entire estimated length of the residual limb and that a reflective angle of 41° provided a nearly perfect perpendicular view of the sides of the limb. For example, the cumulative estimates as the viewing angle changes from 3° to 14° is highly linear (the Pearson Product-Moment correlation (r) was 1.0) and, the linear distances are approximately equal across equal changes in viewing angle.

By contrast, compare the findings for a reflective angle of 41° with comparable estimates for angles considerably less than 41° (e.g., 20° and 30°—which yield relatively greater distances for smaller viewing angles) and for angles considerably greater than 41° (e.g., 50° and 60°—which yield relatively smaller distances for smaller viewing angles). Mirror angles significantly greater or less than 41 not only produced non-equal distance estimates (i.e., greater variability) but also different absolute values (note that the distance values tend to be greater in both cases.

The 2-D image produced using the devices, apparatus, systems, and/or methods described herein provides 3-D surface information about the object (e.g., a viewed object such as a residual limb). In some instances, a 2-D image, map, or projection of the object is used to identify location(s) emitting a higher or increased level of infrared irradiation, which is indicative of increased temperature and which could indicate increased blood flow in that region. In some instances, a 2-D image, map, or projection of the object is used to identify location(s) emitting a lower or decreased level of infrared irradiation, which is indicative of decreased temperature and which could indicate poorer blood circulation in that region. In some embodiments a single two dimensional image represents a large portion of the surface of the object. In some embodiments a single two dimensional image represents the entire surface of the object. In some instances, the camera utilizes a fisheye and/or standard lens. In some instances, a reflective surface is employed to reflect radiation emanating from the object that is not directly in view of the camera. In some instances, the reflective surface is concave or angled such that reflection is directed toward a camera or other monitoring device. In some instances, the reflective surface is spherical, conical, parabolic, etc. In some instances, the reflective surface comprises different segments, such as a conical surface more distant from the apex of the viewing chamber or reflective surface (e.g., such that when the viewing distance, viewing angle, and angle of the reflective surface are properly adjusted, a less-distorted and nearly longitudinally-perfect perpendicular view of the sides of the object are observed), and one or more additional segments closer to the apex of the viewing chamber (e.g., a second segment near the base of the viewing chamber that is more spherical or a second conical segment which has an increased reflective angle relative to the more peripheral conical surface), the purpose of any such additional segments being to capture more of the surface of the viewed object facing away from the camera (from the “opposite side” of the viewed object).

FIG. 1 provides a simple demonstration of the basic approach. On the left is a photograph of a standard Rubik's cube, and on the right is how that cube appears when placed inside a representative concave reflective viewing chamber. The visual information about the sides of the cube is available (albeit, somewhat distorted), as well as undistorted information about the side of the cube closest to the camera. Using such a “distorted” image, and with additional knowledge of the shape and size of the object and the shape of the reflective surface and its distance to the camera, mathematics can be used to determine the actual physical locations for any particular site of interest (point or region) on the actual object.

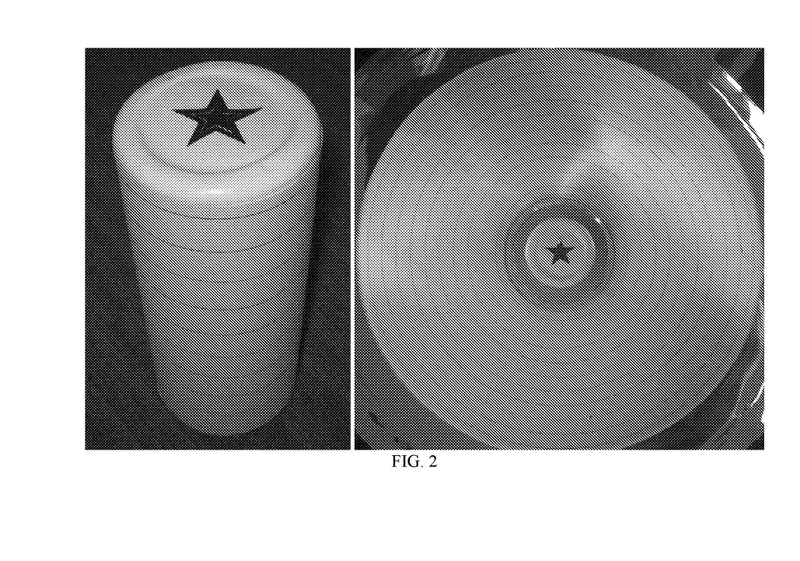

Similarly, FIG. 2 provides a second illustration of this approach. On the left side is a standard photograph of a ‘test cylinder’ which has a star-shaped decal centered on its flat end and circumferential latitudinal lines drawn on its outer wall which are equally spaced along its longitudinal axis. On the right side is a photograph of how that same test cylinder appears when positioned in the middle of a representative conical reflective viewing chamber. Note that the cone's apex angle (angle of the cone's reflective surface relative to the focal axis—the line from the viewer or camera lens to the apex of the conical viewing surface), the frontal focal length (distance from the viewing eye/camera lens to the cone's apex), and the camera's viewing angle have been jointly adjusted to produce an accurate perpendicular view of the outer walls of the test cylinder (i.e., the reflected concentric circles are equidistant apart, as they are on the actual cylinder). This example represents a nearly longitudinally-perfect perpendicular reflected view of the entire exterior surface of the walls of the test cylinder such that for any given ray drawn from the apex of the cone to the outer edge of the viewing surface (i.e., in FIG. 2, any line drawn from the center of the star to the outer perimeter of the cone), equal vertical distances on the test cylinder correspond to equal vertical distances on the reflected portion of the ray. Hence, using proper scaling corrections, the vertical (longitudinal) location of any particular site of interest on the actual test cylinder can be very accurately estimated from its relative radial distance on the reflected conical surface. In addition, the circumferential (latitudinal) location of any such site of interest on the actual cylinder's curved outer wall can be accurately estimated by measuring the angle of the reflected site relative to some standard reference line (e.g., the line that passes through the center of the star and one of its five tips), yielding a 0°- to 359.9° estimate of its angular location. Alternatively, if a fiducial marker or markers are positioned at standardized known locations on the outer curved surface of a cylinder with known diameter, then the site of a reflected point or region of interest on the reflected 2-D image can be compared to the site(s) of the closest reflected fiducial(s), and common trigonometry and transposition used to determine that same site on the actual cylinder.

Note that in both FIG. 1 and FIG. 2 the side of the geometric solid facing the camera is captured without reflection or distortion. Also note that the side opposite to the side facing the camera is not captured in either FIG. 1 or FIG. 2. Certain embodiments of the present invention are directed to devices and methods that modify the shape of the reflecting surface in order to capture more of the “opposite side” of the object. For example, a reflective surface that is linearly conical in the region closest to the camera (as in FIG. 2), but then curves inward at the bottom of the object (assuming the object is elevated relative to the apex of the cone), would capture more or even most of the opposite side of the object. Bringing the object closer to the viewer/camera (e.g., by placing the object on a small pedestal, transparent shelf, or suspending it with string) also allows more of the opposite side of the object to be captured in a single 2-D image.

Certain embodiments of the present invention are directed to devices and methods that provide imaging technology that allows two 2-D images to capture most or all of the entire 3-D surface of a viewed object. In the simplest of such embodiments, a single 2-D image is captured of an object such as illustrated in FIG. 1 and FIG. 2, and then the orientation of the object is reversed (e.g., vertically rotated 180°) and a second image is obtained from that same perspective (e.g., in FIG. 1 the Rubik's cube turned upside down so that the orange matrix which is not visible in FIG. 1 is the side closest to the camera in the second image).

The 3-D imaging technology may use any type and/or multiple types of electromagnetic radiation that can be reflected and captured in one or more image(s). As non-limiting examples, visible spectrum light and Infrared energy (IR) are readily reflected. Materials that reflect electromagnetic radiation are known in the art. Some non-limiting materials that reflect IR include aluminum, aluminum foil, gold, and thermal-reflective Mylar film. In some instances, one type of electromagnetic radiation is used. In some embodiments, materials are used that reflect multiple wavelengths, such as, but not limited to, IR and visible light. Use of multiple types of electromagnetic radiation can provide the benefit of capturing 3-D information from multiple energy dimensions. This information can be mixed/overlaid to facilitate interpretation. For example, in healthcare applications, the photographic and thermographic images of an affected portion of the body can be combined or overlaid to help the healthcare provider interpret the image. Non-limiting examples of materials that reflect both IR and visible light include highly polished aluminum.

In some instances, the best shape for the reflecting surface depends upon multiple factors, such as, but not limited to, the size and shape of the object to be viewed, the amount and location of the surface of the object that is desired to be captured in one or more images, the computational power and/or mathematical ability to render a 3-D representation from the shape, the ability to provide a longitudinally-perfect or nearly longitudinally-perfect perpendicular view, the distance from an apex of the reflecting surface to the camera, etc. Non-limiting examples of shapes of the reflecting surface include concave or angled conical, spherical, parabolic, etc. surfaces. In some instances, the reflective surface comprises portions or segments with different shapes. Non-limiting examples include a conical surface portion more distant from the apex of the viewing chamber and an increased reflective angle conical or spherical surface portion that is closer to the apex of the viewing chamber. In some instances, the surface of the object to be viewed that is facing away from the camera is placed at or near the horizontal plane that is at the same vertical level as the junction of two differently shaped portions of the reflecting surface; in this way, information about the opposite surface of the object can be more easily discriminated and processed (because the image processing software can be provided the “junction angle” at which the two viewing surfaces diverge—with information from angles greater than that junction angle related to the “side view” of the object and information from angles smaller than that junction angle related to the “rear view” of the object).

In some instances, landmarks on the object, features of the object, or added marks or markers are used to guide or determine the location of a particular point of interest or to undistort the 2-D images of the reflected surface(s). In some instances, mathematical equations are used to calculate the location of a particular point of interest or to undistort the 2-D images of the reflected surface(s). In some instances, a computer is used to calculate the location of particular points of interest or to undistort the 2-D images of the reflected surface(s). In a non-limiting example, a 2-D thermal image that displays much/most of the 3-D surface of an object may be used to provide particular locations of relatively high or low temperature.

In some instances, the computer uses an image processor to undistort the 2-D image and/or provide at least one spatial orientation other than the spatial orientation of the camera to the object. In certain aspects the rendered image can be manipulated in three dimensions. In some instances, given additional information about the size and shape of the object or the distances from the camera to different sites reflected from the object, digitized photographic or thermal 2-D images can be mined to generate more natural and intuitive “virtual” views of the object using special image processing software. For example, the 2-D image of the Rubik's cube in FIG. 1 or the cylinder in FIG. 2 may be used to create corresponding virtual 3-D images of those objects, providing the observer with more natural and intuitive views from an unlimited number of spatial orientations. In such cases, the observer would be provided a control device/strategy for manipulating the relative distance and spatial orientation of the object.

In some instances, the custom image processor comprises a specific operation that is applicable to any object that is the subject of the 2-D image in providing 3-D information about the subject. When the image processor is a specific operation, the shape of the viewing chamber, the camera, and the lens system may be held constant and/or the imaging processing may be able to take into account differences in at least one of those properties.

Some non-limiting advantages of using the apparatus and methods disclosed herein are that the 2-D image(s) require(s) less space for storage and transmittal of the 3-D information, taking one or a few images is more efficient than taking a larger number of images to capture the 3-D information of an object, capturing one or capturing fewer images is much faster than taking more images, the apparatus is more simple and less likely to break down (e.g., in some embodiments there are no moving parts) relative to other possible methods (e.g., using a robotic arm to rotate a camera to different orientations around the object), and because fewer images are required, transmittal and processing of the 2-D image(s) to provide a 3-D image or a variety of viewing angles can be performed quickly. Further, using custom image processing software, one or more 2-D images may be used to produce a video that pans the object from a variety of angles and distances; or alternatively, allows a human user to manually redirect the viewing distance and perspective as desired. Notably, the space required to store a 2-D image is small enough that the image could easily be embedded in or attached to emails, text messages, or included in websites, electronic books, advertisements, catalogs, etc. For research, medical, and a variety of other possible applications, the viewers of such 3-D images which have been recreated from the 2-D representation(s) could be allowed to modify and save the image (e.g., a physician might want to circle a region or draw arrows on the image before sending it to the patient, a colleague, or medical students). Another promising application is using the information to facilitate ordering a part during a maintenance task. For example, two-dimensional drawings or photographs in a catalog can be deceiving, instead while using a virtual image embodiment, workers using an online supply catalog could rotate and view a candidate replacement part from a variety of angles to confirm, for example, that there are three mounting holes in a particular configuration located on the base for attachment.

While FIG. 2 shows that, given the proper angle for the reflective surface, camera distance, and viewing angle, a nearly longitudinally-perfect perpendicular view of the object is possible, FIG. 3 shows that the reflected image is not perfect with regard to latitudinal dimensions, but rather is systematically distorted with the circular markers nearer the camera being “magnified” relative to the markers of the same size which are further from the camera. Such distortions also are present in FIG. 2, but are less visually detectable because unique lateral features have been eliminated except for the thickness of the lines—and those are visually negligible. Using special image processing procedures, a less distorted “panoramic” perpendicular view can be created. In both FIG. 2 and FIG. 3 the reflected surface information along any specific ray (i.e., line from the apex of the reflective cone to the outer edge of the reflective cone), is longitudinally accurate, so one such method is to systematically “draw” such a single ray (as depicted as line “A” in FIG. 3), record the values of the pixels along that ray, store the pixel values as a row or column in a data matrix, reposition the ray so that it goes through the apex but passes through the outer edge of the reflective cone at a point that is moved one pixel in either direction (clockwise or counter-clockwise), collect and record the pixel values for that new ray in the next row/column of the matrix, and continue this procedure until returning to the original ray. Depending on the resolution of the 2-D image, the resulting data matrix might have to be compressed or expanded to produce an accurate representation (i.e., the rows compressed or expanded if each extracted ray was entered into the data matrix as a row and the columns compressed or expanded if each extracted ray was entered into the data matrix as a column). Compression or expansion can be accomplished by applying any of a variety of possible techniques, such as, but not limited to, averaging techniques—especially those used in image processing to minimize granular distortions or “pixilation.” The resulting image provides a panoramic view of the external surface of the viewed object. Another related method is to use a pie-shaped section of the image based on two rays separated by a constant angle (e.g., “x”-degree slices—as depicted as section “B” in FIG. 3), average the pixel values for different constant distances along that slice, use those values in the 2-D array, and repeat the procedure moving clockwise or counter-clockwise around the entire image. If the object being imaged is a perfect cylinder, then either the linear or angular ray techniques described above will create a relatively high-fidelity panoramic perpendicular view of the sides of the cylinder; if the object has different thicknesses, then the surfaces of the object that are further from the reflective surface will be magnified relative to the surfaces of the object that are nearer to the reflective surface. In the event a spherical or other non-linear concaved surface is used instead of a conical surface, then similar adjustments may be mathematically applied to both longitudinal and latitudinal aspects, as appropriate for the shape of the concaved surface utilized. Certain embodiments of the present invention include methods for removing distortions from the raw 2-D images by using special 3-D imaging, modeling, or simulation software to create a panoramic view of the object's surface.

### Size and Shape Information

In some aspects, size and shape information is added to the surface information. Most of the above discussion describes how the apparatus and methods described herein are used to capture surface information from a 3-D object and display/store it in two dimensions. By adding information about the shape and size of imaged object, a variety of potentially useful applications are made possible. Non-limiting examples of such applications are those in which the surface information from the 2-D image(s) is “wrapped” to the external surface of either a virtual object (e.g., created with special 3-D graphical simulation software) or an actual object. As discussed above, such representative virtual models of objects could be very useful in many settings because, with custom viewing software, the virtual object could be independently manipulated and viewed from a number of different perspectives (e.g., by a potential customer in a marketing setting, by a player in a video game setting, by a participant in a virtual environment, by a health care professional in a medical setting, etc.). It also would be cost-effective and efficient to produce such representations because they can be based on a single “viewable” file format which contains surface, shape, and size/distance information.

Given that it would be useful to combine an object's 3-D surface information (which is extracted from the 2-D image[s]), with that object's size and shape information, there are a number of ways that the corresponding shape/size information can be obtained. This information can be obtained by any means known in the art. In one aspect, the information is obtained by direct measurement. As suggested above, using trigonometry and interpolation it may be especially simple to assign the surface information (e.g., color) of a specific site to its corresponding location on the surface of a virtual or real object if the object is a simple geometric form or is composed of simple geometric forms, their size(s) known, and there are landmarks available. This also is a plausible strategy in some real-world applications; for example, amputees' residual limbs usually are cylindrical or conical, and common landmarks are often available; thus, special 3-D imaging, modeling, or simulation software can apply trigonometry and interpolation to transfer the surface data from a 2-D image generated by the apparatus and methods disclosed herein to a 3-D representation. If there are not enough visible natural landmarks on the object's surface to create an accurate representation of the object's shape and size, then salient landmarks may be applied to the surface (e.g., painted marks, decals, tacks, etc.), or landmarks may be projected onto the surface of the object using, for example, external laser or light projector(s).

The Rubik's cube shown in FIG. 1 is another example of how a 3-D representation can be created based on simple geometric forms. It is known that its shape is cubical and that it measures 7 cm on each side, and that there are visible landmarks available (e.g., the eight cube corners or the corners of the 56 small squares). Thus, image processing software can be used to create a virtual 3-D model of that “cubical” object and then custom software used to transfer the visual surface information from the 2-D image(s) to its corresponding location on the 3-D representation by applying trigonometry and interpolation using the landmarks as common reference points. If visible landmarks are not available, then fiducial markers can be physically attached to the object at critical sites, or alternatively, points, lines, shapes, images, etc., can be projected onto the object using a laser or other light projector. Certain embodiments of the invention involve using special 3-D imaging, modeling, or simulation software to add surface information to a virtual object that is in the shape of a simple geometric form or which is composed of simple geometric forms by utilizing landmarks or markers located at common known sites in both the 2-D image(s) and the virtual object. If there are not enough visible natural landmarks on the object's surface to create an accurate representation of the object's shape and size, then salient landmarks may be applied to the surface (e.g., painted marks, decals, tacks, etc.), or landmarks may be projected onto the surface of the object using, for example, external laser or light projector(s).

In addition to wrapping surface information to a virtual object, the surface information extracted from the 2-D image(s) can be “wrapped” to the surface of a physical object (e.g., a scaled replica of the originally imaged object which has been carved or constructed using a technique such as 3-D printing, selective laser sintering device, etc.). In such applications, the surface information contained in the 2-D image(s) would be extracted from the image(s) and then transferred to the physical object using an appropriate manufacturing procedure (e.g., robotically controlled paint application). Certain embodiments of the invention involve adding surface information to a real object, which may include objects which have been fabricated.

Certain embodiments of the invention involve combining surface information from the resulting 2-D image(s), such as, but not limited to, that including most or all of the entire 3-D surface information for an object, with a virtual object's shape and size information which has been derived by using a light-field camera. Light-field cameras are capable of estimating distance to different parts of a viewed landscape or object. In some instances, if the distance estimates for enough known landmarks are available, then a virtual model of the object can be created using special 3-D imaging, modeling, or simulation software and the surface information from the 2-D image(s) can be applied to that model by applying trigonometry and transposition using the landmarks as reference points. If there are not enough visible natural landmarks on the object's surface to create an accurate representation of the object's shape and size, then salient landmarks may be applied to the surface (e.g., painted marks, decals, tacks, etc.), or landmarks may be projected onto the surface of the object using, for example, external laser or light projector(s).

Certain embodiments of the invention involve combining surface information from the resulting 2-D image(s), such as, but not limited to, that including most or all of the entire 3-D surface information for an object, with a representative object's shape and size information that has been derived by using a 3-D scanner or similar technology to create a representative model of the actual object. Using special 3-D imaging, modeling, or simulation software, landmarks located at common known sites in both the 2-D image(s) and the representative model of the object are used as reference points when transferring the surface information from the 2-D image(s) to the external surface of the representative model. If there are not enough visible natural landmarks on the object's surface to create an accurate representation of the object's shape and size, then salient landmarks may be applied to the surface (e.g., painted marks, decals, tacks, etc.), or landmarks may be projected onto the surface of the object using, for example, external laser or light projector(s).

Certain embodiments of the invention involve combining surface information from the resulting 2-D image(s), such as, but not limited to, that including most or all of the entire 3-D surface information for an object, with a representative object's shape and size information that has been derived by exploiting parallax after capturing two or more images of the object from different radial perspectives (e.g., before and after moving the camera a known distance left, right, up, down, etc., a known distance). Changing the viewing/camera angular perspective alters the viewing angles of each landmark on the viewed object and the amount of angular change in addition to other known information about the viewing chamber (e.g., if conical, the angle of the cone, the distance from the apex to the observer/camera), can be used to estimate it's perpendicular distance from the focal axis (i.e., “thickness”) at that point. If enough landmarks are analyzed, the shape and the size of the viewed object can be modeled using special 3-D imaging, modeling, or simulation software, and the surface information from the 2-D image(s) then fitted to the external surface of the representative model using the landmarks as reference points and by applying trigonometry and transposition. If there are not enough visible natural landmarks on the object's surface to create an accurate representation of the object's shape and size, then salient landmarks may be applied to the surface (e.g., painted marks, decals, tacks, etc.), or landmarks may be projected onto the surface of the object using, for example, external laser or light projector(s).

Certain embodiments of the invention involve combining surface information from the resulting 2-D image(s), such as, but not limited to, that including most or all of the entire 3-D surface information for an object, with a virtual object's shape and size information that has been derived by capturing two or more images of the object from different distances, another form of parallax (e.g., before and after moving the camera toward or away from the object a known distance). Changing the viewing/camera distance alters the viewing angles of each landmark on the viewed object and the amount of angular change in addition to other known information about the viewing chamber (e.g., if conical, the angle of the cone, the distance from the apex to the observer/camera before and after the move), can be used to estimate it's perpendicular distance from the focal axis (i.e., “thickness” at that point). If enough landmarks are analyzed, the shape and the size of the viewed object can be modeled using special 3-D imaging, modeling, or simulation software, and the surface information from the 2-D image(s) then added to the representative model using the landmarks as reference points and by applying trigonometry and transposition. If there are not enough visible natural landmarks on the object's surface to create an accurate representation of the object's shape and size, then salient landmarks may be applied to the surface (e.g., painted marks, decals, tacks, etc.), or landmarks may be projected onto the surface of the object using, for example, external laser or light projector(s). In one, non-limiting example, the relative changes in the viewing angle of selected landmark locations between two 2-D images can be used to determine landmark location's “thickness” (perpendicular distance from the focal axis to the object's outer surface at the site of the landmark on the object's surface) by using the following equation:

$$T = \frac{\begin{matrix}R \cdot ( V - \frac{X \cdot V}{X + Z} - \frac{X \cdot Z \cdot V \cdot W}{X + Z} - \\ \frac{Y \cdot U}{X + Y} + Y \cdot S \cdot U - \frac{{Y}^{2} \cdot S \cdot U}{X + Y} )\end{matrix}}{S \cdot ( R - \frac{1}{S} )} \qquad Equation 2$$

Where: T=“Thickness” of the object at a specific landmark (perpendicular distance from focal axis to a specific viewed landmark on object's surface); A=Reflective surface angle; B=Observed distant angle; C=Observed near angle; X=Tan (A); Y=Tan (B); Z=Tan (C); U=Distance from apex of viewing-surface angle to distant viewer's location; V=Distance from apex of viewing-surface angle to near viewer's location; R=Tan((2*A)+C); S=(Tan(90−B−(2*A))); and W=(Tan(90−(2*A)−C)).

Certain embodiments of the invention involve combining surface information from the resulting 2-D image(s), such as, but not limited to, that including most or all of the entire 3-D information for an object, with a representative object's shape and size information that has been derived by using a camera but capturing two or more images of the object from the same perspective, without moving the camera, but with the camera's lens system set to be focused to different known distances for each image, and then using common image processing procedures designed to determine the extent that a particular landmark is in focus or not in focus for a given focal distance (e.g., the extent that well defined edges appear at that site). The more such image distances are obtained, the better the resulting model. In some embodiments, the focal distances captured by the camera are in a range from the maximum reasonable distance possible, for example, the greater of (i) the farthest distance from the camera to the object being directly viewed or (ii) the maximum total reflected distance possible when the distance from the camera to the site on the reflected surface is added to the distance from that site to the point in focus located on the focal axis, to the minimum reasonable distance, for example the lesser of (i) the shortest distance from the camera to the object being directly viewed or (ii) the minimum total reflected distance possible when the distance from the camera to the site on the reflected surface is added to the distance from that site to the point in focus located on the “thickest” part of the object that can be placed inside the viewing chamber (where thickness is measured by the perpendicular distance from the focal axis to the surface of the viewed object). In some instances, the camera lens system is systematically adjusted to change the focus and take images at multiple focal lengths. After the virtual model is created, then special 3-D imaging, modeling, or simulation software is used to add the surface information from the 2-D image(s) to the representative model using the landmarks as reference points and by applying trigonometry and transposition. If there are not enough visible natural landmarks on the object's surface to create an accurate representation of the object's shape and size, then salient landmarks may be applied to the surface (e.g., painted marks, decals, tacks, etc.), or landmarks may be projected onto the surface of the object using, for example, external laser or light projector(s).

Disclosed herein are also apparatuses, systems, and methods for assessing a 3-D object and related imaging technology configured for medical uses, in particular fitting and assessment of prosthetics and orthotics, as well as monitoring disease states and conditions. In certain aspects the disease state or condition is associated with aberrant blood flow or inflammation. In some embodiments, the imaging system is used to image a portion of or an entire subject or patient. The subject may include, but is not limited to, a mammal, a dog, a cat, a farm animal, a horse, a primate, an ape, or a human. The portion of the subject or patient imaged may include any portion of the subject or patient including, but not limited to, a head, a face, a nose, an ear, a finger, a hand, an arm, a breast, a back, a torso, a toe, a foot, a knee, a leg, a pelvic region, a lower extremity, a lower torso, a residual portion of an amputated portion of the subject or patient, or an amputated portion of a subject or patient.

In some aspects, thermography is used. In some aspects thermography is used to identify early signs of skin irritation that include lesions, inflamed portions, relatively cooled portions, and/or relatively heated portions, etc. of the subject or patient. In a non-limiting example, thermography is used to identify “hotspots” relative to the surrounding skin temperature in thermographs of an amputee's residual limb that show where skin irritation is beginning. In some instances, the identification is done before the irritation is visible with the human eye. Such sites may indicate where a prosthesis or orthosis can be modified to create a better fit.

In another non-limiting example, thermography is used to identify “cold spots” relative to the surrounding skin temperature in thermographs of an amputee's residual limb that show where there is poor blood circulation. Knowledge of such sites may enable one to avoid skin issues of medical concern; for example, persistent or excessive pressure from the prosthesis or orthosis on a region of skin could prevent blood from reaching those sites (ischemia), possibly leading to significant tissue damage or even necrosis. This may be especially beneficial in dysvascular amputees because they often have neuropathy and are unable to sense such sites. In some instances, relatively cold areas may be detected very early, e.g., after wearing their prosthesis or orthosis a few minutes or walking a few meters, which may allow adjustment of the prosthesis or orthosis before the patient leaves the clinic.

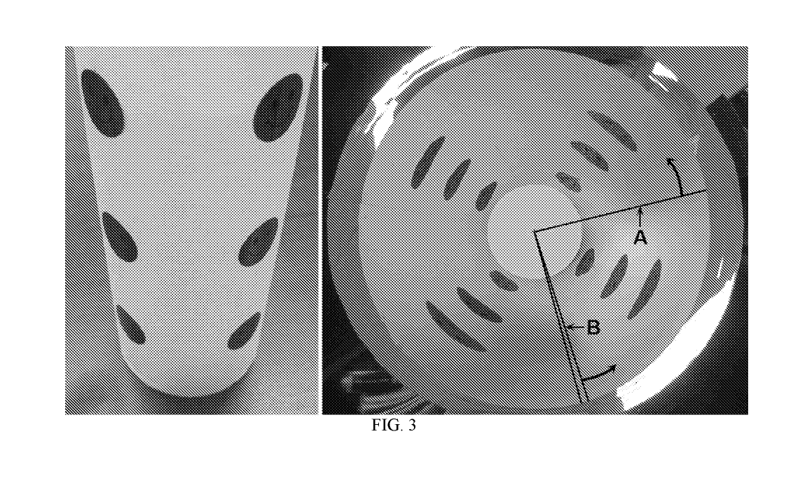

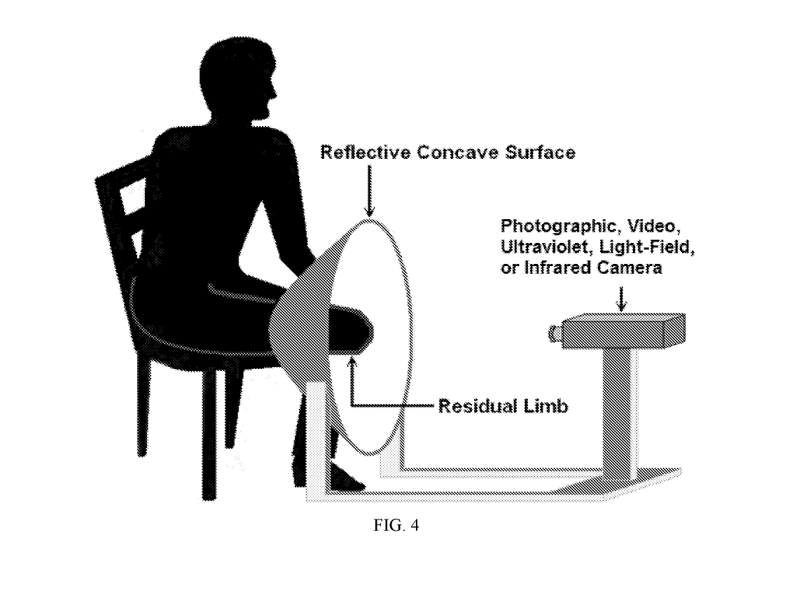

A non-limiting representation of this approach is shown in FIG. 4, which demonstrates a system for recording 3-D information of an amputee's residual limb with one 2-D image. In this case, an inability to include the view from the “opposite direction” is not an issue. Also, note that in some embodiments, the residual limb can be imaged using both a standard photographic camera and an infrared camera, with both cameras capturing images from the concave viewing surface which is composed of a material which is reflective of both IR and visible light wavelengths.

In another non-limiting example, thermography is popularly used in maintenance or quality-control settings to identify “hotspots” in electronic devices and/or components (see FIG. 4—see Example 1 below). Excessive heat can mean there is a short or that something is wrong with the device and that it is about to fail. Placing a thermo-reflective concave viewing surface around such a device allows a greater level of accuracy and information for such remote inspections, helping workers identify the site of the excessive heat more accurately and helping them remotely examine portions of the electronic device not otherwise in view (e.g., on the side, top, bottom, or on the back of the device). In this example, a utility company can, without requiring a worker to use a ladder, get out of their vehicle, or even be present, remotely “inspect” a transformer mounted on a utility pole by remotely capturing one or more thermographic image(s) of the electronic device and/or component and the thermo-reflective concave viewing surface. The image(s) may be captured by any means known in the art, such as by a worker, a drone, or even a satellite for example if the viewing surface is oriented to reflect energy skyward.

### EXAMPLES

### Example 1

### Improving Quality Control and Maintenance of Electronic Devices

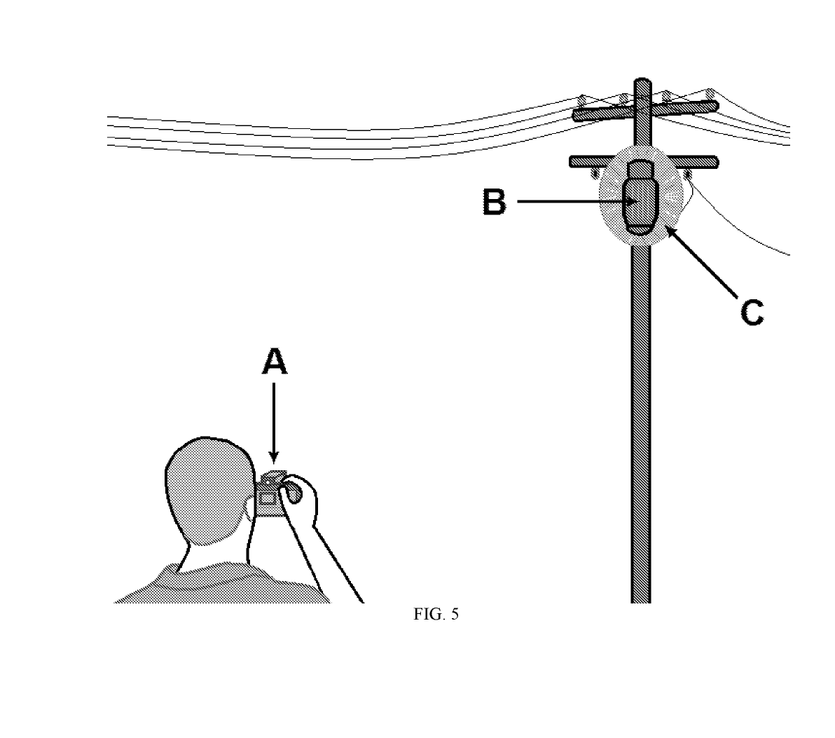

As shown in FIG. 5, a concaved (e.g., conical) surface that has a surface made of a material that reflects infrared energy (C) can be positioned around and behind a transformer (B) on a utility pole. A utility worker can use an infrared camera (A) to view the transformer and surrounding reflective surface from a distance. Any available “zoom” feature on the camera can be used to magnify the view. The viewing location of the utility worker and the orientation of the reflective surface behind the transformer can be pre-set such that (a) a large percent of the transformer's entire surface is captured by the camera and (b) the location of any point of interest (e.g., hot spot) in the resulting 2-D thermal image, can be used to determine the location of that site on the surface of the transformer. With training, utility workers possibly could use their “unaided eye” to determine the presence of excessive heat and the general location of any such sites by viewing the 2-D thermal image captured. Alternatively, the 2-D thermal image captured by the worker may be relayed to a central facility which has image-processing software capable of more precise determinations. In either case, the 2-D thermal image can be stored for future reference and used to provide documentation about the inspection (e.g., by storing the time and date on the image).

### Example 2

### Capturing 3-D Information in One or More 2-D Images to Create a Virtual 3-D Model

Obtaining Raw Surface and Size/Shape Data Using 2-D Image(s)—

A physical object such as a figurine that is colorfully painted with elaborate details can be placed in a viewing chamber which utilizes a conical reflective surface so that its major longitudinal axis falls on or near the focal axis (the line from the camera lens to the apex of the conical viewing surface). Lighting can be provided in a way that illuminates the entire surface of the object, minimizes shadows (e.g., reduces the probability that shadows are interpreted as “landmarks” in the following discussion), and is not directly or indirectly (e.g., reflected off the cone's surface) in the field of view of the camera. In this example, a conical reflective surface can be used, the angle of the reflective surface relative to the focal axis is known, the distance from the camera to the apex of the conical reflective surface is known, the viewing angle of the camera has been set to capture the entire reflective surface, and the cone's angle and distance to the camera have been set to provide a nearly perfect perpendicular view of the sides of an object positioned in the viewing chamber. The bottom of the figurine base can be facing away from the camera, so capturing the surface information from the “other direction” probably is not important, and a single image is obtained that will be used to extract the 3-D surface information. However, in this example, a 3-D model of the figurine can also be created, so because the precise measurements of the figurine (shape and size) are not known, one of the methods described above will be used to estimate the shape and size of the figurine.

For purpose of illustration the method involving the comparison of landmarks at two camera distances is described. The following discussion is intended to provide a representative, understandable and non-limiting description of that procedure. The actual procedures may differ considerably with, for example, additional detailed steps which are beyond the scope of this discussion (e.g., steps involving common image, graphics, modeling, or simulation software algorithms which may be added to “smooth” the resulting virtual model or “blend” the surface colors to make them appear less pixelated). To obtain information about the size and shape of the object, a second image is obtained after the distance between the camera and the surface/figurine has been modified to another known distance toward or away from the object (e.g., the camera or the reflective surface/figurine is moved forward or backwards, in this example the distance between the camera and the reflective surface/figurine is shortened for the second image). Because the figurine's surface has detailed painting, it is presumed that there are sufficient inherent visible “landmarks” available for which distance estimates will be obtained. If the imaged object did not have sufficient visible landmarks, then two additional images can be obtained—in both of those images, latitudinal “concentric circles” (or some other distinctive pattern[s]) can be projected or placed onto the object's surface at different longitudes, with one image obtained with the camera at the same distance as the first image, and the second image with the distance between the camera and the object adjusted to a second known distance, and those two images would be used in the following discussion.

Deriving Size/Shape Data and Merging it with Surface Information.

There are a number of different procedures which can be used; the following steps are intended to provide a non-limiting example of a representative procedure. Taking advantage of parallax the two images can be assessed using special image processing software. Although either image could provide the starting point, in this example the first image (the image obtained at the further camera distance) can be used to extract the surface data and as the reference image for extracting size and shape data. Because the viewing chamber is conical in this example, linear rays (e.g., “A” in FIG. 3) or angular rays (e.g., “B” in FIG. 3) can be used in the following procedures. Selection of the first ray is arbitrary, but for systematic analysis, selection of subsequent rays proceeds clockwise or counterclockwise until returning to the first ray. The direction of the analysis of a given individual ray also is arbitrary; it could start at the apex of the reflective cone and move toward the outer edge of the viewing surface or, as in this example, it could move from the peripheral edge of the viewing surface toward the cone's apex—located on the focal axis. In this example, such inward analyses means that the top of the object (head on the figurine) will be assessed first and the feet/base of the figurine assessed last. The currently selected ray first can be analyzed for the presence of “landmarks.” A landmark is defined as any “section” (radial segment of the ray comprising one or more pixels) on that ray that differs significantly from a preceding section. The definition of section size (e.g., number of pixels included), and the definition of “differs significantly” are determined based on a number of considerations (e.g., the amount of detail present on the object, resolution of the camera, complexity of the object's shape, etc.). The “salience” of a landmark (e.g., the difference in color between the preceding section and the current “landmark” section) also can be determined for all identified landmarks. For each ray, the locations (along that ray) of all identified landmarks, their pattern (e.g., average hue in preceding segment and average hue in “landmark” segment), their angular location (angle of that segment relative to the focal axis), and salience can be stored for use in the subsequent analysis of the second image. Analysis along a given ray can be conducted more than once, for example, using different segment sizes, rules for establishing differences, etc. A distinctive marker may be placed on the “top-most point” of the object (e.g., top of the figurine's head—salient enough and positioned so that it appears as an outer circle when reflected off the wall of the conical viewing chamber), to facilitate locating where to begin analyzing rays. At the other end of the ray, the longitudinal location of the current segment relative to the maximum value (corresponding to the widest portion of the figurine) can be monitored “on the fly” in order to determine the point at which analysis of that ray should stop before entering the non-reflected central region of the image (the upper surface being viewed directly by the camera).

During the second analysis (e.g., involving analysis of the second image which was obtained with the camera closer to the object), each ray can be systematically assessed using the same starting location, sequence, and direction that were used in the first analysis. For each ray, the first landmark identified in the corresponding ray from the first image can be “sought” in the second image. In this example, the camera was moved forward (toward the object) for the second image, so all landmarks for a given ray will occur closer to the periphery in the second image. Consequently, because the analysis moves from the peripheral border toward the apex, the landmark can be sought prior to their location on the first image; indeed, their location in the first image can mark the end of the search process for that landmark. The amount that a given landmark “moves forward” is related to its “thickness” (the perpendicular distance from the focal axis at that longitude to the landmark on the object's outer edge), and by applying trigonometry, the object's thickness at that point can be determined (as defined in Equation 1 above), and added to the other information for that landmark (i.e., its longitudinal location, angular location, and color—all which may be determined in the analysis of the first image). In addition to being bounded on the proximal end of the search by the location of the landmark on the first image, those searches also can be bounded on the distal end of the searched ray by the distance corresponding to that associated with the thickest portion of any object which could reasonably be viewed in the viewing chamber. For the first ray, after the first landmark has been detected and documented, the second landmark is sought, etc., until all landmarks on that ray are documented; then the next ray can be assessed using the same procedure. After all rays have been so analyzed, the basis for the 3-D model exists—a set of 3-D points in space along with their associated color. In this example, the three-dimensional coordinates for each landmark can be determined by their location on a specific ray relative to the central focal axis; specifically, (a) its longitudinal distance from the apex of the viewing surface (e.g., related to its height from the base of the figurine), (b) its latitudinal angle relative to some arbitrary reference ray (e.g., the rotational angle from the ray drawn from the focal axis through that landmark relative to the ray drawn from the focal axis to some arbitrary starting point—perhaps the nose on the face if the figurine), and c) the perpendicular distance from the central focal axis to the landmark (e.g., its “thickness” at that point—as derived by analyzing the discrepancy between two images obtained at different distances by utilizing Equation 1). Image processing software then can be used to position all landmarks in a 3-D space, draw lines between neighboring points (creating the outer “hull” of the object), paint the landmarks on the outer surface (i.e., using the color data stored with each landmark), and then use trigonometry and interpolation to more accurately “paint” the color information available from the first 2-D image to the surface created by the landmarks. Additional graphics and 3-D simulation algorithms can be applied to “smooth” the resulting outer surface or blend the surface colors to help reduce pixilation. Further routines can exploit the surface information available from the non-reflected central portion of the image, applying it to the 3-D representative model that has been created.

Once the 3-D model has been created and its surface information added, then its format could be translated to a form that is consistent with existing conventional 3-D simulation “viewer” software, which would allow a user to spatially “manipulate” the virtual object. In this example, the figurine could be viewed from different orientations (e.g., tilted, turned, brought closer, etc.), using common user human interface device(s) (e.g., keyboard, joystick, mouse, touchscreen, etc.). Alternatively, the format could remain in a unique form and a custom viewer created which would similarly allow a user to manipulate the virtual object. The resulting virtual model also may be converted to a format compatible with 3-D printers, allowing an actual object to be created. The corresponding color information may be applied to the surface of the 3-D printed object using appropriate 3-D color applicators (e.g., robotically controlled paint applicators).

### Example 3

### Prediction of Sites of Irritation on an Patient's Residual Limb

One non-limiting clinical application of the imaging system disclosed herein involves identifying sites of potential skin irritation or poor blood flow on an amputee's residual lower limb. As depicted in FIG. 4, this application can involve inserting the residual limb of a lower-limb amputee through a hole in the center of a concave reflective surface made of polished aluminum, so that it reflects both infrared and visible light. The reflective surface can be convex with respect to the patient but concave with respect to a visible light and/or thermal imaging camera that is positioned to directly image the distal end of the inserted limb and image the sides of the residual limb indirectly by capturing the side views of the limb being reflected off of the concave reflective surface. Fiducial markers which are either warmer or colder than skin temperature can be attached to the patient's residual limb at relocatable landmark sites (e.g., one marker centered on the anterior tibia, 2 cm below the lower edge of the patella, and three more at that same longitude, but at points which, starting with the first marker, are attached at latitudes which are 25%, 50%, and 75% of the entire limb circumference at that longitude).

Different concave shapes can be used for the reflective surface (e.g., conical, spherical, parabolic); in this example, a conical surface is used because (a) as discussed above and shown in FIG. 2, by properly adjusting the angle of a conical reflective surface and the distance from its apex to the camera, a nearly longitudinally-perfect perpendicular side view can be obtained of the surface of the patient's residual limb; (b) relatively straight forward trigonometry can be used to translate the location of a point or region of interest found on the 2-D image back to its location on the patient's leg; and (c) as discussed above, if desired, a panoramic view of the patient's leg can be created by systematically combining angular rays. If areas of potential interest (e.g., relatively hot or cold regions) are detected in the 2-D thermal image, their location on the 2-D image can be used to pinpoint the location of the corresponding regions on the patient's limb, either by transposition based on their relationship to the nearest fiducial markers, or by applying trigonometry as discussed above, to determine the longitudinal distance from the apex to the region of interest and its latitudinal location, as determined by the angle of rotation from a common reference line (e.g., the line that passes through the center of the image and the anterior edge of the remaining tibia). For confirmation and additional analysis, an identified site then can be scanned using conventional thermography or scanned using a conventional 2-D laser-Doppler scanner. If both thermal and laser-Doppler images are obtained for the same region of interest, then cross-correlations can be performed to determine the extent that the two types of imaging technology (i.e., conventional 2-D thermograph and conventional laser-Doppler 2-D image), depict similar patterns at that site.

The 3-D images can be taken before and after a short walk using a new or adjusted prosthesis. If any areas are detected which are measurably different (hotter or colder) after walking than before walking, then the prosthetist is informed, shown the locations, and, depending on the prosthetist's expert opinion, possibly perform modifications to the prosthetic socket at that time, before the patient leaves the clinic. If only areas of moderate concern are found, then they can be documented (precise location, temperature, size, etc.) and the patient asked to return for a follow-up visit. On the return visit, if the initial measures (before walking) for those sites of interest identified during the earlier visit indicate significantly different temperatures, or if those same regions worsen after the patient completes a walk, then the prosthetist can be notified and corrective alterations applied to the prosthesis. The same general procedure could be used for a patient receiving a new orthotic device.

### Example 4

### Testing Prototype Imaging System

Unilateral transtibial amputees were tested to provide an initial feasibility test for the new apparatus and method described herein. The primary research questions were whether the new imaging system could capture all or most of the surface area of an amputee's residual limb in a single 2-D image; whether regions of possible irritation (ROI) could be detected in the 2-D image; whether any such identified ROIs could be validated by LD images of peripheral blood perfusion at those sites; and, for the amputee's sound foot, whether there were relationships among (a) thermal images of the bottom of the sound foot, (b) peak plantar pressure maps obtained while subjects walked, and (c) LD images of the bottom of the sound foot.

Subjects.

Approval to conduct the proposed research with human subjects was obtained from the University of Texas Health Science Center at San Antonio (UTHSCSA) Institutional Review Board and the South Texas Veterans Health Care System's (STVHCS) Research Committee. Subjects were two volunteer unilateral transtibial amputees with new or newly refitted prosthetic limbs. Demographic information was collected (items D1 to D8 in Table 1) and relevant anthropometrically-related measures recorded (items A1 to A10 in Table 1).

Apparatus.

Subjects already had their own new or recently refitted prosthetic limbs. A Tiger4 Pro thermal imaging camera and software manufactured by Teletherm Infrared Systems was purchased with grant funds and used in combination with the novel viewing chamber described above. With this camera, there was no way to view the current scene until after the image was taken, so a small laser was attached to the top of the camera to help align the camera with the center of the viewing chamber. In addition, the location of the tripod holding the camera was marked on the floor to help insure the correct distance and camera angle were maintained. The 2-D laser Doppler imaging system (camera, computer, and software) used was a PIM 3 Laser-Doppler Scanner Imager manufactured by Perimed. This system simultaneously captures 2-D images of both blood perfusion and light intensity.

TABLE 1
Demographic and anthropometric information for the two participants.

| Variable | Subject 1 | | Subject 2 | |
|---|---|---|---|---|
| D1. Sex | Male | | Male | |
| D2. Age | 29 | | 37 | |
| D3. Cause of amputation | Traumatic | | Traumatic | |
| D4. Currently diabetic | No | | No | |
| D5. Laterality of | Left | | Right | |
| amputated limb | | | | |
| D6. Time since | 2 | years | 4.8 | years |
| amputation | | | | |
| D7. Phantom-limb pain in | None | | None | |
| last month | | | | |
| D8. Phantom-limb pain | None | | None | |
| now | | | | |
| A1. Height | 172.7 | cm | 165.1 | cm |
| A2. Weight | 122.5 | kg | 79.4 | kg |
| A3. Length of sound foot | 27 | cm | 24 | cm |
| A4. Width of sound foot | 11 | cm | 10 | cm |
| A5. Length of residual | 21.5 | cm | 14 | cm |
| (mid-patellar tendon to | | | | |
| tibial end) | | | | |
| A6. Knee circumference | 46.5 | cm | 36.3 | cm |
| A7. Leg circumference at | 38 | cm | 31.8 | cm |
| proximal row of fiducials | | | | |
| A8. Leg circumference at | 37 | cm | N/A | |
| middle row of fiducials | | | | |
| A9. Leg circumference at | 33 | cm | 30 | cm |
| distal row of fiducials | | | | |
| A10. Number of fiducials | 12 | | 8 | |
| used | | | | |

The only modification made to the LD system was that a platform was built to which the camera arm was secured (because all images were of the lower limbs, the camera needed to be closer to the ground). The system used to capture plantar pressure measures while subjects walked was a Pedar Sensole System manufactured by Novel. A small laser was attached to the top of the thermal camera to facilitate positioning/aiming the camera before an image was taken. A platform was built so that the LD camera could be located closer to the ground. Fiducial markers were attached to the subject's residual limb at different landmark sites which could be relocated on the subject's second session. The primary criteria for the markers were that they be safe and easily identifiable in both the thermal and LD (light intensity) images. Several different types of markers were investigated and ranged from warm to cool (relative to skin temperature). Warm fiducials tested included button battery-powered LEDs (which were found to not emit enough heat for easy recognition) and wire coils (Kanthal 34 Gauge AWG A-1 and AWG36 0.1 mm 138.8 Ohm/M Nichrome Resistor Resistance Wire)—which were ruled out because it could not be assured that the temperature would not exceed a safe level. Cool fiducials tested included a variety of rubber, silicon, felt, and other synthetic materials, cooled in a refrigerator freezer, and transported to the test site in an insulated/cooled container. The final markers selected were made from common glue sticks, which were 1.1 cm in diameter and sliced to a thickness of 0.4 cm. This material retained its relatively cool temperature for an adequate amount of time and was visible on both thermal and LD (intensity) images. The fiducials were attached to the limb of the subject by a double-sided adhesive tape.

Procedure.

During an initial 20-min rest period, a standardized procedure was used to determine and mark (using a surgical marking pen) the future location of 8 or 12 (8 were used if the residual limb was shorter than 16 cm) fiducial markers, based on anatomical landmark sites (i.e., the mid-patellar tendon, the distal end of the residual limb, and the tibial crest (line formed by the anterior-most edge of the remaining tibia). Using the tibial crest as a reference line, a first marker was positioned 5 cm below the mid-patellar tendon, a second marker positioned 3 cm proximal to the end of the residual limb, and a third (if the residual limb was longer than 16 cm), was positioned midway between the first two markers. Next, the circumference of the residual limb at each of those markers was measured and three other markers positioned at equal distances around the circumference at those points (i.e., in the horizontal plane—at medial, lateral, anterior, and posterior sites). Also during the initial rest period, a short “pain survey” was administered aurally. In this survey, subjects were asked four yes/no questions as to whether they were experiencing (a) any pain on the surface of their residual limb; (b) any irritation on the surface of their residual limb; (c) any pain on the bottom of their sound foot; and (d) any irritation on the bottom of their sound foot. In the event that a subject answered affirmatively to any question, then (a) the subject was asked to rate the pain/irritation on a scale of 1 to 9 where 1 is slight pain and 9 is extreme pain; (b) the subject was asked to point to the site of the greatest pain/irritation and that site was recorded (relative to the markers); and (c) the subject was asked if there was a second site for pain/irritation (if so, the subject pointed to it and its location was recorded).

At the end of the initial 20-min rest period, a “3-D thermograph” was taken using viewing chamber as described herein; a standard photograph also was taken of the residual limb in the viewing chamber. In addition, 4 standard thermographs were taken using medial-, lateral-, anterior-, and posterior-views; a thermograph also was taken of the bottom of the subject's sound foot. Next, with the fiducials still in place, LD images were taken of the residual limb (medial, lateral, anterior, and posterior views along with a distal-to-proximal view of the end of the residual limb). An LD image also was taken of the bottom of the subject's sound foot. Subjects were required to wear protective glasses during all LD measures (to help insure the laser used in the LD did not accidentally strike their eyes).

After the first battery of thermal and LD images were obtained, subjects were fitted with Pedar shoe inserts on both their sound and prosthetic foot. This system was used to collect peak plantar-pressure measures while subjects then walked at their own self-selected speed for 50 meters (a figure-8 route was used which included 4 left turns and 4 right turns). Time to complete the walk was recorded. Also at the completion of the walk, the standard clock time was recorded, the prosthesis was removed, the leg was towel dried, and the 8-12 fiducial markers reattached to the residual limb. Next, a second battery of images were collected which included a 3-D thermograph using the novel viewing chamber and a standard thermal image of the bottom of subject's sound foot. Importantly, the “hottest” location identified in the 2-D image of the residual limb was identified on the subject's residual limb and designated a primary “region-of-interest” (ROI). Using the ROI as a center, a rectangular “template” then was used to mark the sites of four fiducial markers, and a conventional 2-D thermograph was taken of that ROI. Next, two conventional LD images were obtained—one for the identified ROI and one of the bottom of the subject's sound foot, and the pain survey was administered a second time.

After the second battery of images were obtained, subjects donned their prosthesis and shoes (both with Pedar inserts) and, no sooner than 20 min after the completion of the first 50 m walk, began a second, 100 m walk (the same course was used but now included 8 left and 8 right turns). After completing the second walk, the same procedure (as that following the first walk) was used to collect a third battery of images. If time permitted, a fourth identical battery of images were collected after a minimum of 20 min following the second walk—i.e., the purpose was to determine if a terminal rest period reduced any detected irritation. Due to time limits (2 hrs), no terminal measures were obtained for the first subject and only a partial set of images were obtained for the second subject.

At the end of the first session, subjects' prostheses were fitted with a pedometer (which recorded their daily activity over the next two weeks) and then scheduled for their next visit two weeks later. On their second visit, the same measurement procedure described above was used with the following exceptions: (1) following the initial rest and 3-D thermograph, appropriate fiducial markers were reinstated at the locations of the corners of the ROI identified during the first session, and conventional 2-D Ir and LD images were taken of the ROI; (2) the post-100 m measures were the last measures obtained, and (3) pedometer data were collected and pedometers removed from the subject's prosthesis.

### A. Results

Subject 1.

Self-Reports of Pain/Sensitivity Before and after Walking.

Subject 1 reported no initial pain or sensitivity in the residual limb or the bottom of the sound foot and no pain or sensitivity after completing the 50 m walk. Following the 100 m walk, Subject 1 reported pain on the residual limb at a point 2 cm proximal to Marker 2 (which was located on the tibial crest 3 cm above the end of the residual limb). The subject rated the level of pain in that region as 3.5 on the 1-9 scale. No other pain or sensitive areas were reported for the residual limb or the bottom of the sound foot during the first session.

Thermal and LD Images of the Residual Limb Before and after Walking.

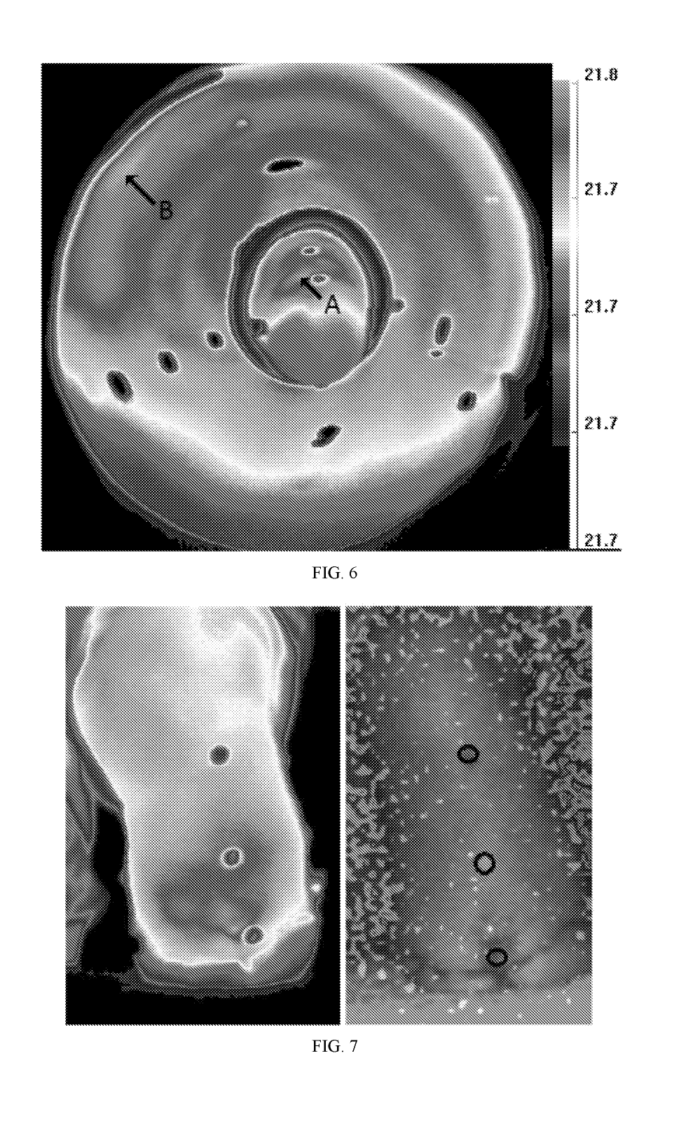

The first 3-D image is shown in FIG. 6. Darker blue and green ovals indicated the fiducial markers. They do not line up perfectly in FIG. 6 because the subject had difficulty positioning and holding his residual limb in the horizontal orientation required in order for all fiducials to line up as. In this case, the limb was oriented slightly downward; for example, a (relatively cooler) green area is the end of the subject's residual limb and the area above the green area and below the inner blue circle (the hole in the end of the cone) the top of the residual limb is visible directly. For example, point A is a directly visible hotspot located slightly to the left of the tibial crest and about one third of the way between the most distal and middle markers on the tibial crest), and region B is the same region as A, but reflected (and magnified) off the conical surface. As shown on the left side of FIG. 7, the initial anterior thermal image shows increased heat in the same region. However, the right side of FIG. 7 shows the corresponding anterior LD image, and while there might be some increased blood perfusion in that region, in general, there is no salient visible evidence for in greatly increased perfusion in that region. There were no obvious areas of increased heat (thermographs) or blood perfusion (LD images) in any of the other three standard thermal and LD image orientations (i.e., medial, lateral, or posterior views).

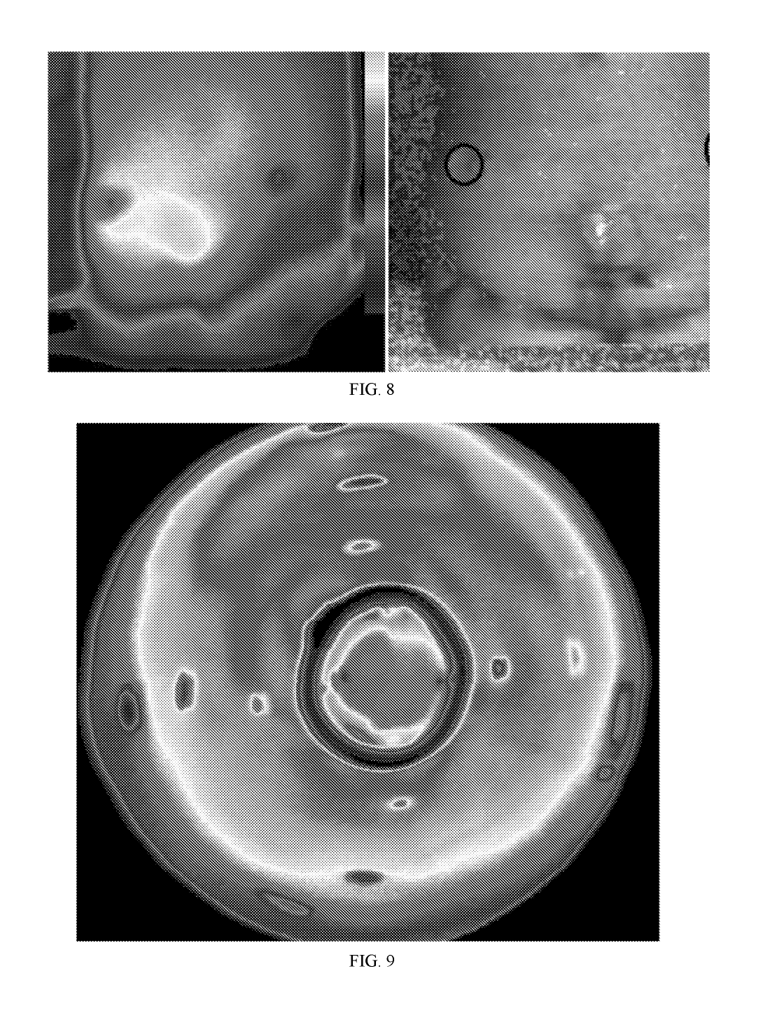

Following the 50 m walk, the same area was evident in the second 3-D image, and was formally selected as the primary region-of-interest (ROI) for subject 1. It should be noted that the subject reported that, while not experiencing pain or sensitivity, he had experienced pain in that region in the past. FIG. 8 shows standard thermal (left side) and LD (right side) images of that ROI following the 50 m walk. The region is salient in the thermal image and one smaller site is evident in the LD image. The mean temperature for a 7×7 pixel area in the center of the hotspot was 22.92° C. and that for a comparable area outside the ROI was 22.82° C.; the mean perfusion level for the most active 7×7 pixel area in the LD image was 63.61 PU and that for a comparable area outside the ROI was 34.13 PU. The image results following the 100 m walk were similar to those following the 50 m walk (shown in FIG. 8).

Walking Speed During the First Session.

Subject 1 completed the 50 m walk in 53.3 s (0.94 m/s) and the 100 m walk in 119.3 s (0.84 m/s).

Session 2

Pedometer Measures of Between-Session Activity.

For subject 1, either the pedometer malfunctioned or the subject did not walk very much; it indicated 113 steps the first afternoon, but activity on the other days ranged from 0 to 47 steps per day.

Self-Reports of Pain/Sensitivity Before and after Walking.

Subject 1 reported no initial pain or sensitivity in the residual limb or the bottom of the sound foot and no pain or sensitivity after completing the 50 m walk. Following the 100 m walk, Subject 1 reported pain on the residual limb pointing to the center of the identified ROI. The subject rated the level of pain as 5 on the 1-9 scale. No other pain or sensitive areas were reported for the residual limb or the bottom of the sound foot.

Thermal and LD Images of the Residual Limb Before and after Walking.

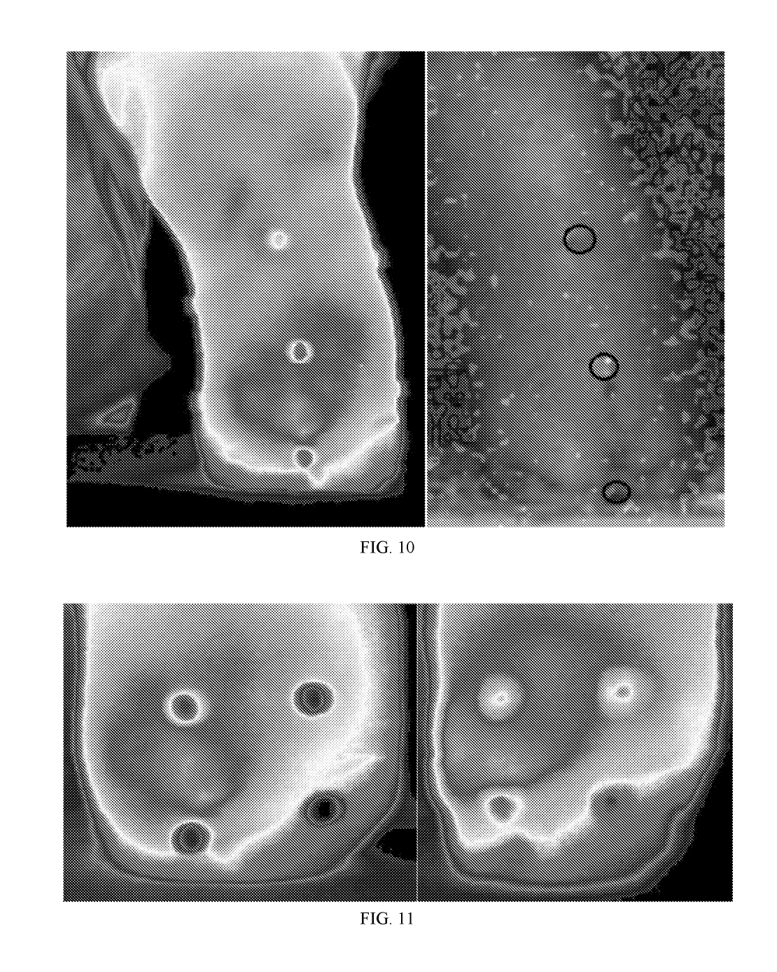

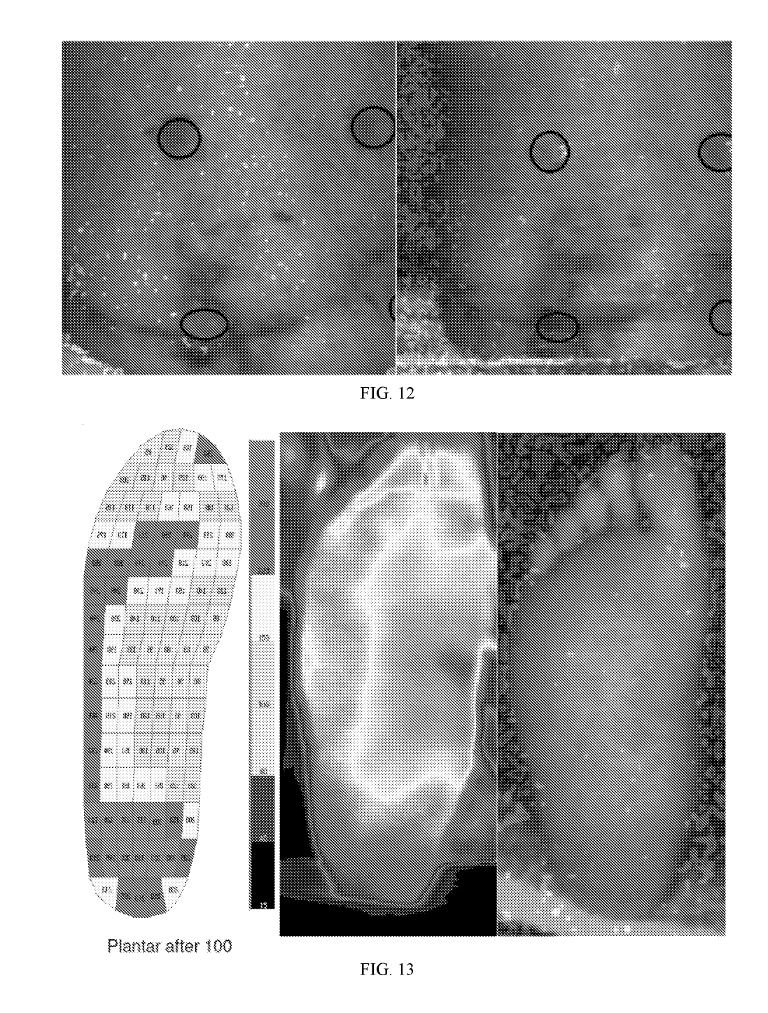

FIG. 9 shows the initial 3-D image for Subject 1 after the initial 20 min rest period during the second session. Similarly, the anterior view for the thermal (left side of FIG. 10) and LD (right side of FIG. 10) show patterns similar to those for the corresponding initial images obtained in the first session—specifically, the identified ROI is salient in the thermograph but not evident in the LD image.

The ROI from Session 1 was relocated, fiducials attached, and thermal and LD images taken of that ROI before any walking during the second session. FIG. 11 shows the thermal image for that ROI before walking (left) and after completing the 100 m walk (right). Following the 100 m walk, the ROI has seemed to broaden and intensify—it was following the 100 m walk that

Plantar Pressures on the Sound Foot while Walking and Associated Thermal and LD Images of the Sound Foot after Walking.

Subjects walked a total of four times during both sessions: 50 m and 100 m in each session. Results were similar across the four walks, and the 100 m walk during the second session was selected to be shown in FIG. 13 below (higher image quality). In FIG. 13, a map of mean peak plantar pressures is shown on the left side, corresponding thermal image following the 100 m walk in the center, and corresponding LD image showing measures of perfusion following the 100 m walk on the right. Note that the plantar pressure map shown on the left (which is generated by the Pedar system) has been horizontally flipped so that in all three images, the reader is viewing the bottom of the foot from below the foot. As shown, there was little correspondence among the three sets of measures. The possible exception is the region around the hallux and second toe (higher pressures while walking, greater heat afterward, and possibly increased perfusion afterward). Indeed, FIG. 13 shows an inversed relationship for the mid-medial area, with lower plantar pressures, corresponding to higher heat, and low blood perfusion.

Walking Speed During Second Session.

Subject 1 completed the 50 m walk in 61.6 s (0.81 m/s) and the 100 m walk in 126.3 s (0.79 m/s); both walks were slower in the second session than they had been in the first session (i.e., 0.94 m/s for the 50 m walk and 0.84 m/s for the 100 m walk).

Subject 2/Session 1

Self-Reports of Pain/Sensitivity Before and after Walking.

Subject 2 reported no pain or sensitivity in the residual limb or the bottom of the sound foot before walking, after walking 50 m, or after walking 100 m.

Thermal Images of the Residual Limb Before and after Walking.

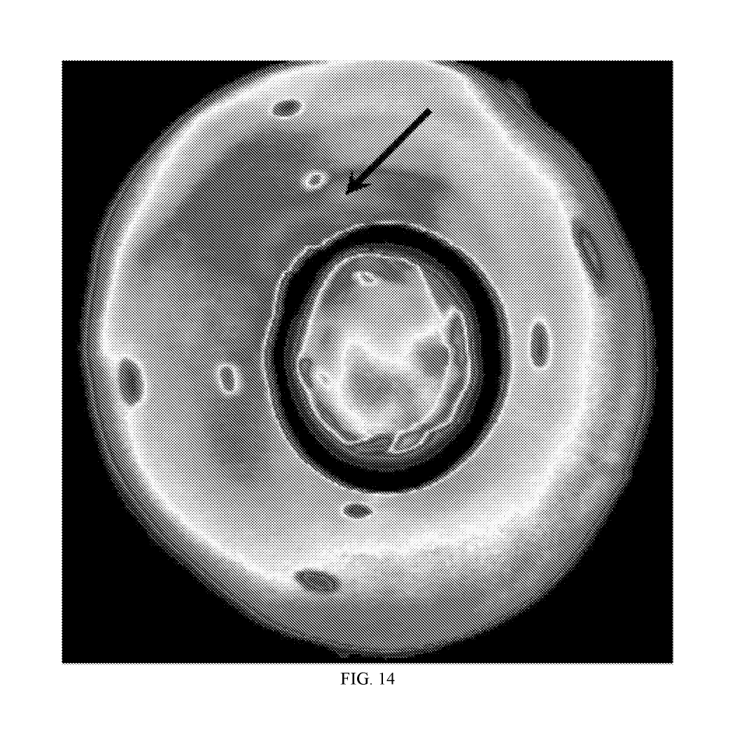

FIG. 14 shows the 3-D image for Subject 2's residual limb after completing the 50 m walk. Based on that image, a ROI was identified which fell approximately on the tibial ridge and 2 cm above the proximal marker on the tibial crest. This ROI was evident in the first pre-walk 3-D image (not shown) as well as in the initial anterior thermal image (shown on the left side of FIG. 15). Unlike the ROI identified for the first subject, the ROI for the second subject was also fairly salient in the first anterior LD image (shown on the right side of FIG. 15).

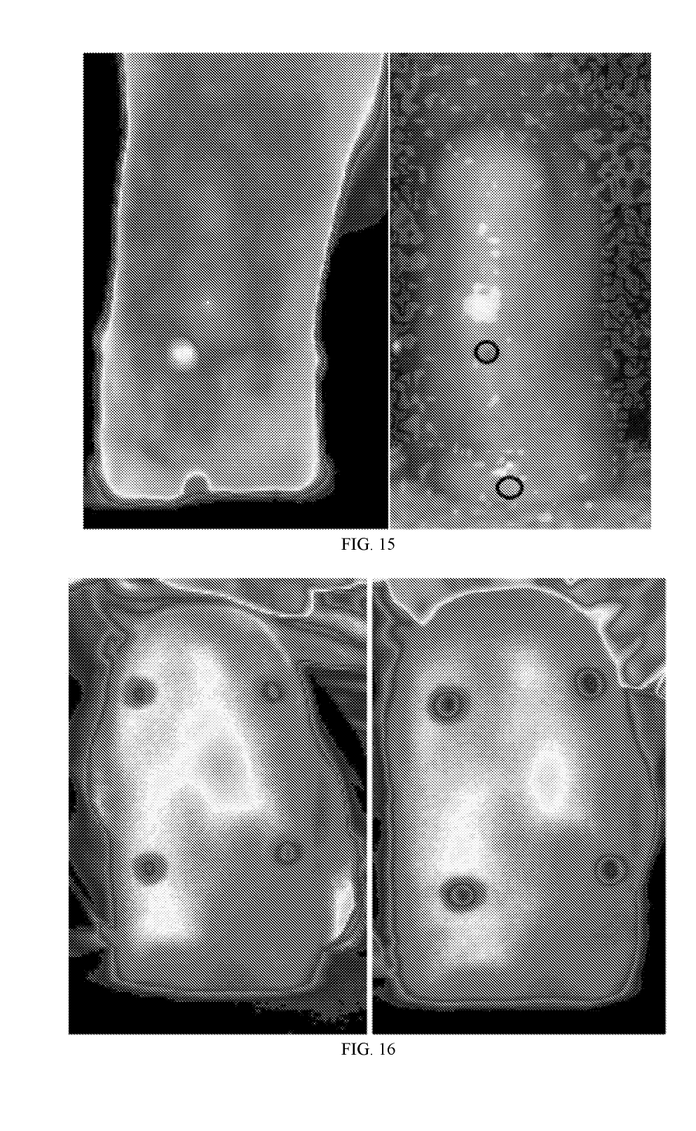

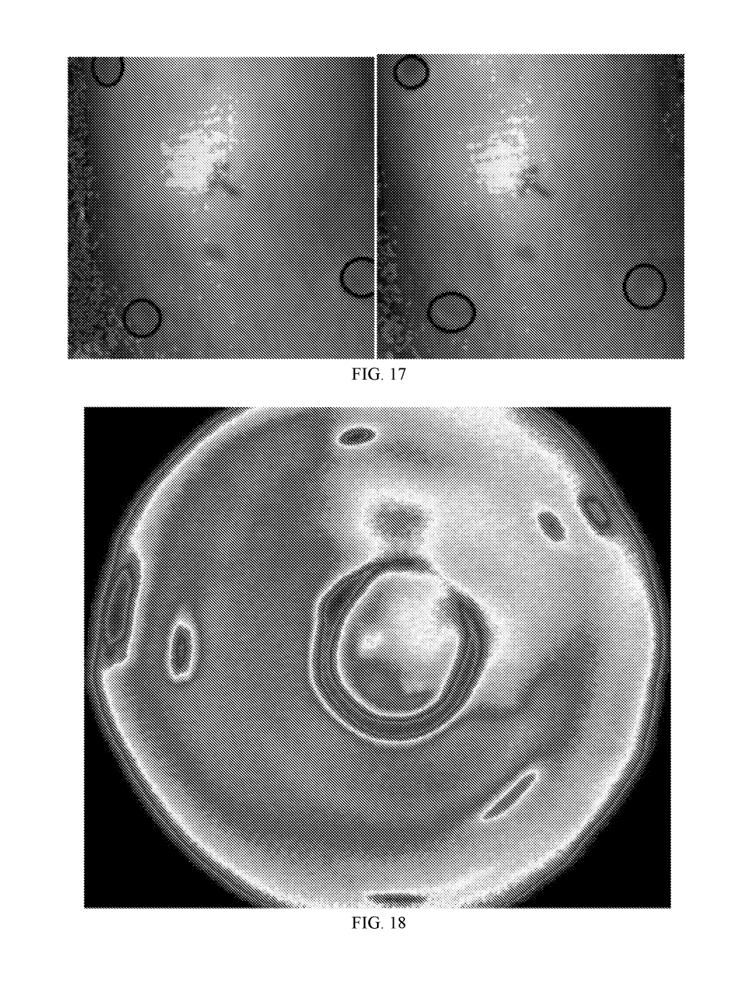

FIG. 16 shows standard thermal images for the identified ROI after the 50 m (left) and 100 m (right) walks. Note that the affected regions appear to be similar in both images. FIG. 17 shows the corresponding standard LD images for the identified ROI after the 50 m (left) and 100 m (right) walks. As in the thermal images, note the remarkable similarity in the area and intensity of increased blood perfusion in the ROI. The mean temperature for a 7×7 pixel area in the center of the hotspot was 23.24° C. and that for a comparable area outside the ROI was 23.23° C.; the mean perfusion level for the most active 7×7 pixel area in the LD image was 132.93 PU and that for a comparable area outside the ROI was 39.74 PU.

Walking Speed During the First Session.

Subject 2 completed the 50 m walk in 51.1 s (0.98 m/s) and the 100 m walk in 97.2 s (1.03 m/s).

Session 2

Pedometer Measures of Between-Session Activity.

The mean number of steps per day for Subject 2 during the two-week period was 1,024 steps per day. There three days where no steps were measured, the median number of steps per day was 1046, and number of steps ranged from 0 to 1,939.

Self-Reports of Pain/Sensitivity Before and after Walking.

Subject 2 reported no pain or sensitivity in the residual limb or the bottom of the sound foot before walking, after walking 50 m, or after walking 100 m.

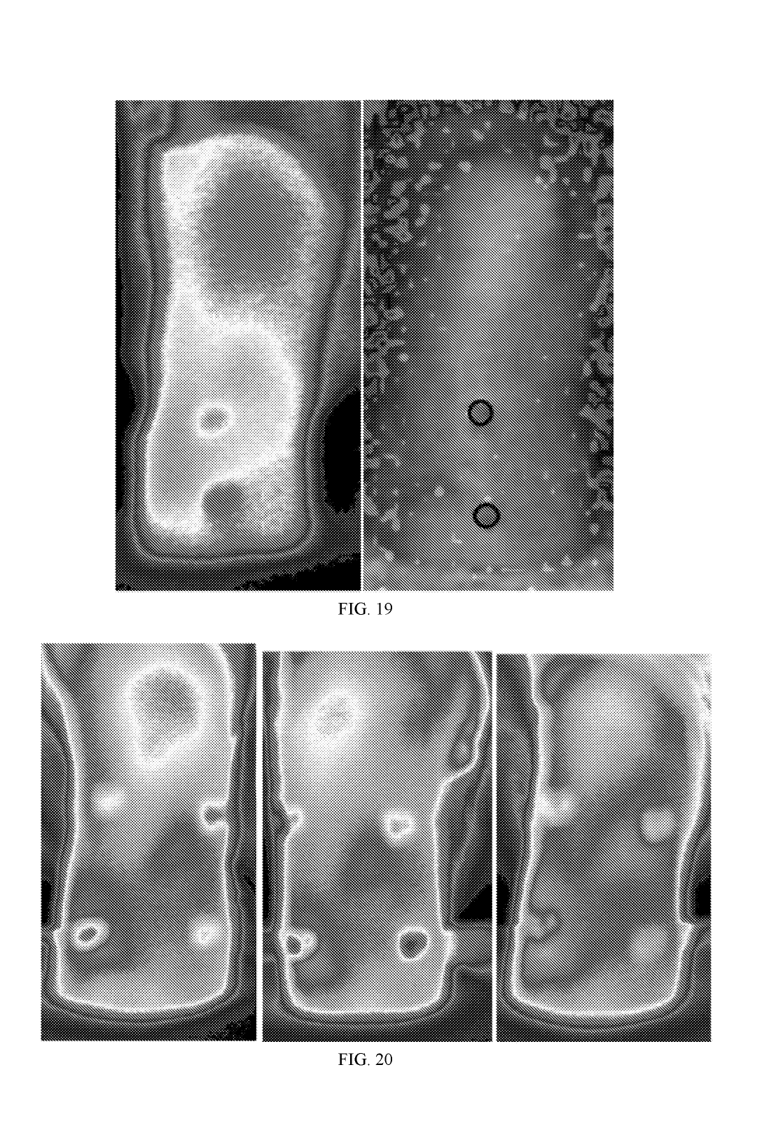

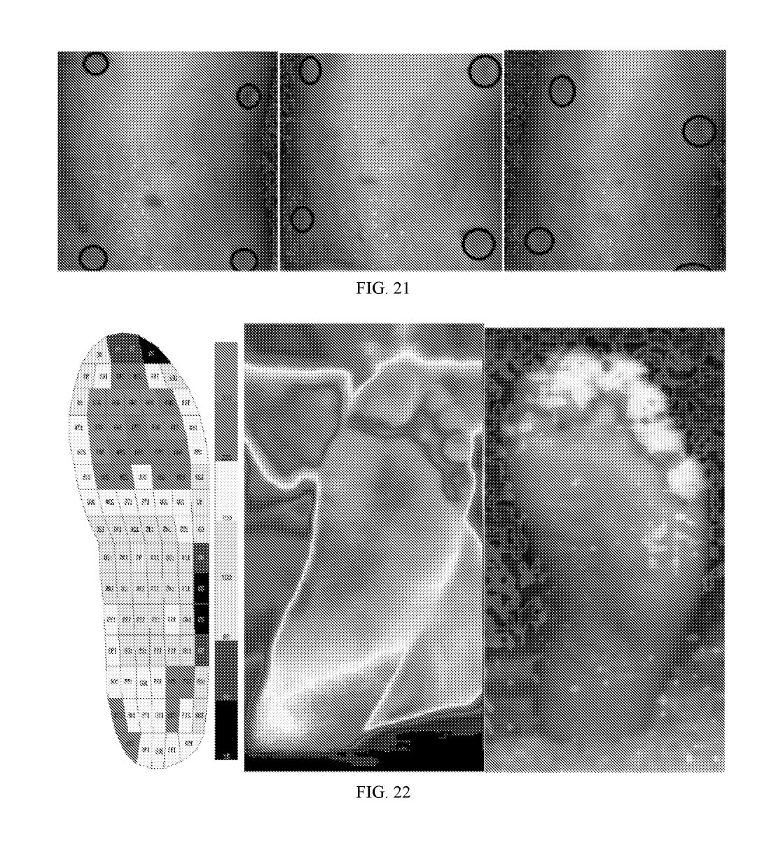

Thermal Images of the Residual Limb Before and After Walking. FIG. 18 shows the initial 3-D image of Subject 2's residual limb. Conspicuously absent is the ROI identified during the first session—if anything, there are increased measures on the opposite (posterior) side of the limb. The disappearance/reduction of the ROI is further evidenced by the thermal (left) and LD (right) standard anterior images taken at the beginning of the second session and shown in FIG. 19. While there is some general thermal and perfusion activity, the concentrated regions evident in the first session (see FIG. 15-17) are absent in the second session. Similarly, while the initial (left), after-50 m walk (center), and after-100 m walk (right) thermographs of the identified ROI shown in FIG. 20 show some increase in activity with increased walking, the increased activity is not concentrated at the original ROI site (i.e., the center of the four markers). In parallel fashion, the initial (left), after-50 m walk (center), and after-100 m walk (right) LD images shown in FIG. 21 do not depict a concentration of activity at the center of the ROI as they did in Session 1 (see FIGS. 15 and 17).

Plantar Pressures on the Sound Foot while Walking and Associated Thermal and LD Images of the Sound Foot after Walking.

Subjects walked a total of four times during both sessions: 50 m and 100 m in each session. Results were similar across the four walks, and the 100 m walk during the first session was selected to be shown in FIG. 22. As in FIG. 13, in FIG. 22 a map of mean peak plantar pressures is shown on the left side, corresponding thermal image following the 100 m walk in the center, and corresponding LD image showing measures of perfusion following the 100 m walk on the right. Note that the plantar pressure map shown on the left (which is generated by the Pedar system) has been horizontally flipped so that in all three images, the reader is viewing the bottom of the foot from below the foot. As with Subject 1, for Subject 2 there was little correspondence among the three sets of measures. Plantar pressures were the highest in the metatarsal heads and heel; thermal measures were highest between the toes and in the mid medial region, and perfusion was salient in the hallux and four toes.

Walking Speed During the Second Session.

Subject 2 completed the 50 m walk in 47 s (1.06 m/s) and the 100 m walk in 90 sec (1.11 m/s).

The results were quite promising. The prototype viewing chamber apparatus and method were effective in allowing the capture of most/all of the surface of an amputee's residual limb in a single 2-D thermal image. The amount of information in one such 3-D image can replace the information in five standard thermographs or LD images (i.e., medial, lateral, anterior, posterior, and distal views), or the device might be useful as a “screening” device for detecting candidate ROIs, which then are followed by more conventional images of those regions.

The developed/tested approach was able to detect regions of possible concern in both subjects, even before their first walk during the first session. For the first subject, the ROI detected in the initial 3-D image was indirectly validated by the subject, who later reported pain at that site, but only after completing the second, longer 100 m walk. On the subject's return two weeks later, the identified ROI was still present and again, was reported by the subject to be painful only following the second longer 100 m walk. It should be noted that during the first session the subject reported having had pain at that site in the past and had been given a shot in that region to help with the pain.

For the second subject, a potential region of concern also was identified in the initial 3-D image, and was subsequently verified by standard thermographs and LD images of that site. Notably, the subject did not report any irritation or pain at that site, suggesting the possibility that the device might be useful as a method for very early detection. Perhaps one of the more interesting and provocative findings, was that the region identified for Subject 2 was “gone” when the subject returned to the lab two weeks later. Although purely speculative, one possible explanation has some empirical support and, if correct, has implications for translational research such as this—especially those involving the collection of measures over longer periods of time.

1. An imaging system for producing a two-dimensional image of a physical object, comprising:
a reflective surface that reflects at least one portion of the electromagnetic spectrum; and
at least one camera facing the reflective surface that is capable of capturing at least one image based on reflected electromagnetic radiation;
wherein (i) the reflective surface is concave in respect to the at least one camera, comprises an apex, and is configured to reflect at least one type of electromagnetic radiation emanating from the surface of a physical object positioned along the principal axis of the reflective surface and (ii) at least one camera is positioned to capture the reflected electromagnetic radiation.

2. The imaging system of claim 1, further comprising a computer based image processor wherein the computer based image processor is configured to determine the location on the physical object that is emitting the reflected electromagnetic radiation received by the at least one camera.

3. The imaging system of claim 1, wherein the concave surface is spherical, conical, or parabolic.

4. The imaging system of claim 1, wherein the concave surface comprises more than one shape.

5. The imaging system of claim 1, wherein the concave surface comprises a conical surface portion more distant from the apex of the reflective surface and an increased reflective angle conical and/or spherical surface portion that is closer to the apex of the reflective surface.

6. The imaging system of claim 1, wherein the concave surface is configured to reflect radiation emanating from physical object along the principal axis and 360 degrees about the principle axis.

7. The imaging system of claim 1, wherein the reflective surface is capable of reflecting more than one type of electromagnetic radiation.

8. The imaging system of claim 1, wherein at least one camera has a fisheye lens.

9. The imaging system of claim 1, wherein at least one camera is capable of capturing the surface image of the object as a single image.

10. The imaging system of claim 2, wherein a computer based image processor is configured to provide a representative view of the object surface, wherein the representative view can be manipulated in virtual three dimensional space.

11. The imaging system of claim 1, wherein the system is capable of capturing the surface image of the object from two or more angles from the principle axis of the reflective surface, from two or more distances from the apex of the reflective surface, and/or using two or more focal distances.

12. The imaging system of claim 2, wherein the computer based image processor is configured to determine and/or assign a size, shape, location, or any combination thereof of a region of interest on the physical object that is emitting the reflected electromagnetic radiation based on the size, shape, location or any combination thereof of a region of interest identified in the captured image.

13. The imaging system of claim 1, wherein at least one camera is capable of capturing multiple types of electromagnetic radiation and/or the imaging system comprises at least two cameras each that are capable of capturing a different type of electromagnetic radiation than the other.

14. The imaging system of claim 1, wherein the at least one type of electromagnetic radiation is infrared light and at least one camera is a thermographic camera responsive to the infrared energy spectrum.

15. The imaging system of claim 1, wherein the concave surface reflects infrared energy.

16. The imaging system of claim 1, wherein the concave surface is aluminum.

17. The imaging system of claim 1, wherein the system is configured to produce an image that is a hotspot map of the object.

18. The imaging system of claim 1, wherein the system is configured to produce an image that is a coldspot map of the object.

19. A computer based image processor capable of mapping a location on an object based on a reflection of the object from a concave reflector captured by at least one camera.

20.-35. (canceled)

36. A method of identifying the location of skin irritation and/or early signs of skin irritation on a subject comprising:
placing a portion of the subject to be imaged, the subject having actively worn a prosthetic or orthotic device, along the principal axis of a reflective concave structure in view of at least one camera connected to an imaging system;
capturing at least one image of reflected infrared radiation emitted from the part of the subject being imaged with the at least one camera;
identifying any region of interest in which skin temperature is higher and/or lower than average skin temperature;
and mapping any such region of interest identified on the captured image to its corresponding actual location on the part of the subject being imaged using a computer based image processor.

37.-53. (canceled)
